(19) United States
(12) Patent Application Publication
(10) Pub. No.: US 2023/0039675 A1
(43) Pub. Date: Feb. 9, 2023

(54) ELECTRON BEAM RADIATION SYSTEM WITH ADVANCED APPLICATOR COUPLING SYSTEM HAVING INTEGRATED DISTANCE DETECTION AND TARGET ILLUMINATION

(71) Applicant: INTRAOP MEDICAL CORPORATION, Sunnyvale, CA (US)

(72) Inventors: Michael F. Turk, Porter Ranch, CA (US); Christopher J. Patane, San Mateo, CA (US); Richard L. Johnson, Suffolk, VA (US)

(21) Appl. No.: 17/779,796

(22) PCT Filed: Nov. 24, 2020
(86) PCT No.: PCT/US2020/061963

§ 371 Date: May 25, 2022

Related U.S. Application Data
(60) Provisional application No. 62/941,327, filed on Nov. 27, 2019.

(51) Int. Cl. G02B 19/00; G02B 27/10

(52) U.S. Cl. CPC G02B 19/0047; G02B 27/1006; G02B 27/14

(57) ABSTRACT

The present invention relates to linear, straight through electron beam machines that incorporate a rotary coupling system to easily attach and manually or automatically rotate field defining members such as applicators and/or shields to the electron beam machines. The rotary coupling systems also incorporate functionality for using different kinds of optical signals to automatically provide illumination, reference mark projection, and/or distance detection. The optical signals generated downstream from heavy collimator components and are transmitted along the central axis of the field defining elements so that function and accuracy are maintained as the components rotate.

### PRIORITY CLAIM

This application claims the benefit of U.S. Provisional Patent Application No. 62/941,327 filed on Nov. 27, 2019, entitled “ELECTRON BEAM RADIATION SYSTEM WITH ADVANCED APPLICATOR COUPLING SYSTEM HAVING INTEGRATED DISTANCE DETECTION AND TARGET ILLUMINATION”, the disclosure of which is hereby incorporated by reference in its respective entirety for all purposes.

### FIELD OF THE INVENTION

The present invention relates to the field of linear, straight through electron beam machines and methods used for therapeutic uses. More particularly, the present invention relates to linear, straight through electron beam machines that incorporate a rotary coupling system to easily attach and manually or automatically rotate field defining members such as applicators and/or shields to the electron beam machines. The rotary coupling systems also incorporate functionality for using different kinds of optical signals to automatically provide illumination, reference mark projection, and/or distance detection. The functionality for using different kinds of optical signals also could be incorporated into any other kind of electron beam machine.

### BACKGROUND OF THE INVENTION

Electron beam (“ebeam”) radiotherapy is a type of external beam therapy in which electrons are directed to a target site on a patient in order to carry out a desired treatment. Features of the electron beam such as energy, dose rate, dose, treatment duration, field size, field shape, distance to the patient, and the like are factors in carrying out treatments.

Electron beam linear accelerator-based machines are one type of electron beam machine used in electron beam radiotherapy. The MOBETRON electron beam machine available from IntraOp, Sunnyvale, Calif., is an example of a mobile, self-shielded, electron beam linear accelerator (LINAC) machine useful in electron beam radiotherapy.

A typical electron beam LINAC machine uses a linear accelerator to accelerate a supply of relatively lower energy electrons. The electrons may be sourced by thermionic emission from cathodes. The electrons are injected into the accelerator and gain energy as they travel down the structure. The power needed to accelerate the electrons often is supplied by magnetrons or klystrons. Downstream of the linear accelerator, the energized electron stream is fed to a collimator. The collimator helps to narrow the beam of electrons such as to cause the electrons to become more aligned in a specific direction as well as to cause the spatial cross section of the beam to become smaller. A collimator also may help to homogenize the beam energy across its cross-section. Downstream from the collimator, one or more additional components may be used to further shape, define, and/or homogenize the beam. Examples of such field defining components include applicators and shields. Applicators or shields may be used singly or in combination.

It is desirable for electron beam machines to have positioning degrees of freedom that include rotation of beam shaping components. For example, it might be desired that an entire collimator be able to rotate at least +/−90°. Some machine designs to not allow rotation to be incorporated into machine function unless cumbersome components are added. For example, some conventional accelerators designed to deliver electrons are also expected to deliver high energy x-rays. The consequence is that the head or collimator is heavy, as it contains either multi-leaf collimators or tungsten collimators to define the X-Y treatment field. Such collimation devices must be thick enough to attenuate the x-ray radiation to 5% or less. The collimation devices also must allow field sizes of 25 to 40 cm at the patient plane. Thus, conventional collimators are too heavy to rotate without motor assistance. The head rotation also is limited due to use of cables needed to run the motors. Rotation can also interfere with how distance detection, illumination, and electron beam aiming strategies can be implemented. Better strategies to incorporate rotation functionality into electron beam LINAC machines are desired.

When used to generate electrons, field defining components such as applicators and/or shields made of plastic or metal, are attached to the collimator. Historically, electron beam LINAC machines may have had either a permanent or detachable mount to accept either electron applicators or x-ray shadow blocks. The wide-spread introduction of multileaf collimators eliminated the need for a shadow block tray attachment, but a detachable mount to attach electron applicators is still required. Without a mount, the electron applicators would be too long and awkward to use. It often is desirable to limit or otherwise define the shape of the electron beam field emitted from an electron beam LINAC machines. One strategy to accomplish this is by placing shields with aperture of appropriate size and shape downstream from the collimator such as at end of the applicator. Better strategies for mounting, de-mounting, and orienting applicators and shields are desired.

It often is desirable to illuminate a treatment site so that electron beam (also “ebeam”) machine can be aimed accurately, so that the progress of a treatment can be monitored, and the like. Some conventional units generally use an incandescent light bulb that is positioned just outside the collimator. When the field light is activated, the light turns on and a mirror is moved in position to reflect the light on the target surface. Because of the relatively large light bulb to target surface distance, there is penumbra of 2-5 mm. The positioning of such a light bulb also can interfere with potential rotational positioning strategies. Better techniques to illuminate target sites without interfering with machine positioning are desired.

Treatments require that the electron beam LINAC machine be positioned at an accurate distance from the treatment site. Distance can affect the dose, ebeam energy, dose rate, and field size delivered to the target site. Some conventional strategies have used distance indicators that are optical projections of a scale. Such a projected scale has the potential to be accurate at the isocenter distance, but is less accurate at shorter and longer distances. Also, such devices can be affected by rotational positioning. Better strategies to measure distance are needed.

### SUMMARY OF THE INVENTION

The present invention relates to linear, straight through electron beam machines that incorporate a rotary coupling system to easily attach and manually or automatically rotate field defining members such as applicators and/or shields to the electron beam machines. The rotary coupling systems also incorporate functionality for using different kinds of optical signals to automatically provide illumination, reference mark projection, and/or distance detection. The optical signals generated downstream from heavy collimator components and are transmitted along the central axis of the field defining elements so that function and accuracy are maintained as the components rotate. The principles of the present invention can be used with respect to any kind of ebeam machine. For purposes of illustration, the principles of the present invention will describe the invention in the context of electron beam LINAC machines.

Rotational capabilities are provided by rotatably mounting field defining members downstream from the collimator. Collimator rotation is not needed, as field size and shape can be established using the field defining members. The rotary coupling system is attached downstream from the collimator and is easily detachable for servicing components located inside the collimator. In illustrative embodiments, the rotary coupling system continues the conical opening of the collimator to improve the homogeneity resulting from wall scattering, finally terminating in a cylindrical section. In many embodiments, cylindrical applicators that attach to the rotary coupling system help to reduce the opening of the distal end of the collimator to the diameter of the applicator that is attached.

The rotary coupling system allows field defining elements to be easily rotated manually or automatically in clockwise or counter clockwise directions. Desirably, the rotation axis may be the same as the beam centerline. Rotation is unlimited in either direction. Rotation can be indexed, though, such as to allow rotation in 2° increments, and the rotation can be locked to secure the applicator position when it is in a desired orientation. The rotation mechanism desirably has a rotary position sensor for feedback purposes.

Derm radiotherapy generally may require 15-25 treatments. The field size used for Derm applications might have shielding inserted at the end of the applicator to protect healthy tissue. Since a patient might not always be on the treatment table in the exact same position each day, applicator and/or shield rotation results in the ability to rapidly position the electron beam to the correct orientation on the patient. Manual rotation is preferable to motorized rotation as it is more reliable (no cables, no motors, no electronics needed), and the manual field defining member(s) can be positioned more rapidly than a motor-driven collimator.

In one aspect, the present invention relates to an electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface; and
c) a rotary coupling system that rotatably couples at least the first field defining member to an upstream component of the electron beam unit such that the first field defining member is rotatable on demand around a rotational axis independent of rotation of the upstream component, wherein the rotary coupling system comprises a through aperture, an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface.

In another aspect, the present invention relates to an electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
   i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
   ii) a tilted minor mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
   iii) a window through which light can be directed at the tilted mirror from a location outside the through aperture of the rotary coupling system; and
d) a light system positioned outside the through aperture of the rotary coupling system, wherein the light system produces a light signal and emits the light signal in a manner such that the light signal comprises light from one or more wavelength bands of the electromagnetic spectrum in the range from 200 nm to 2000 nm and is aimed at the tilted mirror through the window in a manner effective to be reflected downstream by the mirror along the linear pathway toward the surface.

In another aspect, the present invention relates to an electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
   i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
   ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
   iii) a window through which at least one optical signal can be directed at the tilted mirror from a location outside the through aperture of the rotary coupling system; and
d) a light system positioned outside the through aperture of the rotary coupling system, wherein the light system produces a light signal and emits the light signal in a manner such that the light signal is aimed through the window at the tilted mirror in a manner effective to be reflected downstream along the linear pathway to the surface through the first field defining member through aperture.

In another aspect, the present invention relates to an electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
   i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
   ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
   iii) a window through which light can be directed at the tilted mirror from a location outside the through aperture of the rotary coupling system; and
d) a light system positioned outside the through aperture of the rotary coupling system, wherein the light system produces a light signal and emits the light signal in a manner such that the light signal is aimed at the tilted mirror through the window in a manner effective to be reflected downstream along the linear pathway through the first field defining member to the surface, wherein the light system comprises a laser light source that produces a light signal comprising a visually observable optical reference mark that is reflected downstream through the first field defining member outlet onto the surface in a manner such that the location of the reference mark on the surface is indicative of how the electron beam is aimed at the surface.

In another aspect, the present invention relates to an electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising a central axis, an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
   i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
   ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
   iii) a window through which at least one optical signal can be directed at the tilted mirror from a location outside the through aperture of the rotary coupling system; and
d) a light system positioned outside the through aperture of the rotary coupling system, wherein the light system produces a composite light signal and emits the composite light signal in a manner such that the composite light signal is aimed at the tilted mirror in a manner effective to be reflected downstream along the linear pathway through the first field defining member toward the surface, wherein the light system comprises:
   i) a laser light source that produces at least a portion of a first light signal comprising a visually observable optical reference mark.
   ii) an LED light source that produces at least a portion of a second light signal comprising visually observable LED illumination; and
   iii) an optical combiner that combines at least the first and second light signals to provide the composite light signal in a manner such that the reference mark is reflected downstream through the first field defining member onto the surface in a manner such that the location of the reference mark on the surface is indicative of how the electron beam is aimed at the surface and such that the LED illumination illuminates the surface where the electron beam is aimed.

In another aspect, the present invention relates to an electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
   i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
   ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
   iii) a window through which light can be directed at the tilted mirror from a location outside the through aperture of the rotary coupling system; and
d) a distance detection system positioned outside the through aperture of the rotary coupling system, wherein the distance detection system comprises a controller, a laser light source, and an image capturing sensor, wherein:
   the laser light source is configured to emit a laser light signal at the tilted mirror in a manner effective to be reflected downstream along the linear pathway through the first field defining member toward the surface such that at least a portion of the laser light signal is reflected from the surface back to a location on the tilted mirror that is a function of a distance characteristic of the surface relative to a distance reference; and
   the image capturing sensor observes and captures image information of the tilted minor, said image information indicative of the location on the tilted mirror onto which the laser light signal is reflected from the surface; and
   the control system uses the capture image information to determine a distance characteristic of the surface with respect to the distance reference.

### BRIEF DESCRIPTION OF THE DRAWINGS

FIG. 1 schematically shows an illustrative embodiment of an electron beam radiation system of the present invention.

FIG. 2 schematically shows more details of an illustrative electron beam generation unit used in the electron beam radiation system of FIG. 1.

FIG. 3 schematically shows an alternative embodiment of an electron beam generation unit useful in the electron beam radiation system of FIG. 1.

FIG. 4 schematically shows an alternative embodiment of an electron beam generation unit useful in the electron beam radiation system of FIG. 1.

FIG. 5 schematically shows an exploded, side cross-section view of the rotary coupling system of the present invention of FIG. 2 in alignment with field defining members in the form of an applicator and a shield.

FIG. 6 schematically shows a side cross-section view of the rotary coupling system of the present invention of FIG. 2 in alignment with field defining members in the foam of an applicator and a shield.

FIG. 7 schematically shows an exploded, side cross-section view of the rotary coupling system of the present invention of FIG. 2 with field defining members in the form of an applicator and a shield mounted to the rotary coupling system.

FIG. 8 schematically shows an alternative side cross section view of the assembled components of FIG. 7.

FIG. 9 schematically shows how components to automatically measure distance can be incorporated into the assembled components of FIG. 8.

FIG. 10 schematically shows how components to automatically illuminate and project reference marks onto a target site can be incorporated into the assembled components of FIG. 8.

FIG. 11 shows a side view of the assembled components of FIG. 8 in more detail.

FIG. 12 shows a perspective view of the assembled components of FIG. 11 with a housing removed to uncover the underlying collimator and rotary coupling system.

FIG. 13 is a further side view of the components of FIG. 8 showing how the applicator and shield (attached to the outlet of the applicator) are mounted and demounted from the rotary coupling system.

FIG. 14 is a bottom, perspective view of the components shown in FIG. 13.

FIG. 15 is a top perspective view of the applicator used of FIG. 11.

FIG. 16 is a top perspective view of the shield of FIG. 11.

FIG. 17 is a side perspective view showing how the applicator and shield of FIG. 11 are mounted to and de-mounted from each other.

FIG. 18 is another side perspective view showing the shield and a lower portion of the applicator of FIG. 17.

FIG. 19 is a bottom perspective view of the applicator and shield of FIG. 17.

FIG. 20 is a bottom perspective view showing the shield and a lower portion of the applicator of FIG. 19.

FIG. 21 is a perspective view of a library including applicators and shields of the present invention.

FIG. 22 shows a top view of the applicator of FIG. 11 wherein cross-section guides B-B and C-C are shown.

FIG. 23 is identical to the top view of FIG. 22 except for showing cross-section guide lines A-A.

FIG. 24 is a side cross-section perspective view of the applicator of FIG. 22 taken along line C-C.

FIG. 25 is a side cross-section perspective view of the shield of FIG. 16 taken along line A-A.

FIG. 26 is a side cross-section perspective view of a portion of the applicator of FIG. 23 taken along line A-A, wherein the button is un-pressed and the shiftable plunger is in a locking position in which the shield is locked on the applicator.

FIG. 27 is a side cross-section perspective view of a portion of the applicator of FIG. 22 taken along line B-B showing the shiftable plunger in a locking position in the pocket behind the ramp in the wide slot.

FIG. 28 is a side cross-section perspective view of a portion of the applicator of FIG. 23 taken along line A-A, wherein the button is pressed causing the shiftable plunger to shift over in the wide slot to unlock the shield, allowing the shield to be removed from the applicator.

FIG. 29 shows a side perspective view of the rotary coupling system of FIG. 2 in more detail.

FIG. 30 shows another side perspective view of the rotary coupling system of FIG. 2 in more detail.

FIG. 31 shows a top view of the rotary coupling system of FIG. 29, wherein cross-a section guide is shown that provide the view of FIG. 35.

FIG. 32 is an exploded view of the rotary coupling system of FIG. 29, wherein an illustrative number of fasteners used to assemble the exploded components also are shown.

FIG. 33 is a side perspective view of the rotary coupling system of FIG. 29 with some components removed to show the underlying components of the rotary indexing system.

FIG. 34 is a close up perspective view of the rotary indexing components shown in FIG. 33.

FIG. 35 shows a cross-sectional side perspective view of the rotary coupling system of FIG. 29 taken along line A-A of FIG. 31.

FIG. 36 is a bottom perspective view of the central core and mirror assembly used in the rotary coupling system of FIG. 29, also showing components of the optical illumination system in optical communication with the mirror.

FIG. 37 is a bottom perspective view of the central core and mirror assembly used in the rotary coupling system of FIG. 29, also showing components of the optical illumination system and the distance detection system in optical communication with the mirror.

FIG. 38 is an exploded view of the central core and mirror assembly of FIG. 37, wherein an illustrative number of fasteners used to assemble the exploded components also are shown.

FIG. 39 is an exploded side perspective view of a portion of the upper sub-assembly of the rotary coupling system of FIG. 29, wherein an illustrative number of fasteners used to assemble the exploded components also are shown.

FIG. 40 is another exploded side perspective view of a portion of the upper sub-assembly of the rotary coupling system of FIG. 29, wherein an illustrative number of fasteners used to assemble the exploded components also are shown.

FIG. 41 is another exploded side perspective view of a portion of the upper sub-assembly of the rotary coupling system of FIG. 29, wherein an illustrative number of fasteners used to assemble the exploded components also are shown.

FIG. 42 is an exploded perspective view of a portion of the lower sub-assembly of the rotary coupling system of FIG. 29 shown in more detail, wherein an illustrative number of fasteners used to assemble the exploded components also are shown.

FIG. 43 is a top view of components of the rotary coupling system of FIG. 29 that provide rotary locking functionality.

FIG. 44 is a bottom perspective view of the button actuated locking device of FIG. 43.

FIG. 45 is an exploded perspective view of a portion of the lower sub-assembly of the rotary coupling system of FIG. 29 shown in more detail, wherein an illustrative number of fasteners used to assemble the exploded components also are shown.

FIG. 46 schematically shows an exploded, side cross-section view of the rotary coupling system of FIG. 7 in which only the shield, and not the applicator, is used as a field defining member downstream from the rotary coupling system.

FIG. 47 schematically shows an exploded, side cross-section view of the rotary coupling system of FIG. 7 in which only the applicator, and not the shield, is used as a field defining member downstream from the rotary coupling system.

### DETAILED DESCRIPTION OF THE PRESENTLY PREFERRED EMBODIMENTS

The embodiments of the present invention described herein are not intended to be exhaustive or to limit the invention to the precise forms disclosed in the specification and Figures. Rather a purpose of the illustrative embodiments chosen and described is so that the appreciation and understanding by others skilled in the art of the principles and practices of the present invention can be facilitated. While illustrative embodiments of the present invention have been shown and described herein, the skilled worker will appreciate that such embodiments are provided by way of example and illustration only. Numerous variations, changes, and substitutions will now occur to those skilled in the art without departing from the invention. No unnecessary limitations are to be understood therefrom. The invention is not limited to the exact details shown and described, and any variations are included that are within the scope of the claims.

All patents, patent applications, and publications cited herein are incorporated by reference in their respective entireties for all purposes.

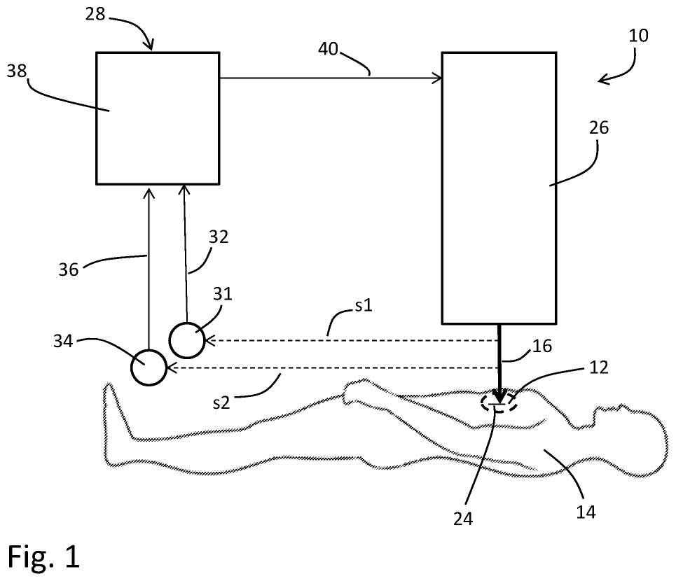

An exemplary embodiment of an electron beam (also referred to as an “ebeam”) radiation system 10 of the present invention is schematically shown in FIG. 1. Electron beam radiation system 10 is useful to irradiate a target site 12 on a patient 14 with a desired electron beam radiation dose in one or more treatment fractions. Unit 26 is aimed so that electron beam 16 contacts and irradiates the target site 12 on patient 14 to deliver the desired dose using an appropriate electron beam energy, dose rate, and/or treatment time.

System 10 is useful for irradiating a wide range of treatment sites anywhere in or on body or body parts of the patient 14. For example, external treatments may involve treating the ears, nose, face, forehead, scalp, back, shoulders, neck, arms, hands, chest, abdomen, pelvic region, legs, or feet. Due to the ability to control the shape and aim direction of the electron beam aimed at the target site 12, system 10 is useful for treating target sites with a variety of shapes and contours.

Due to its compact nature, self-shielding capabilities, and/or mobility in many modes of practice, system 10 may be used to apply electron beam radiation before or after surgery. In some applications, such as scar amelioration, it is beneficial to irradiate the closed incision promptly. For example, system 10 can be used to deliver electron beam radiation dose(s) in a time period ranging from 0 to 24 hours, or even 0 to 5 hours, or even 0 to 1 hour, or even 0 to 30 minutes of the time of a surgery. This ability to apply irradiation treatments promptly is contrasted to treatments that use very large and immobile machines housed in separate, heavily-shielded environments that are remote from the treatment location. Radiation treatment in such large, remotely housed machines has been applied post-operatively after a delay of hours or days, thereby missing the opportunity to achieve the optimal benefits of electron beam radiation therapy.

System 10 is useful to carry out a wide range of treatments for which electron beam irradiation provides a treatment, benefit, or other desired effect for surgery or as an adjunct to surgery or other procedure. For example, system 10 may be used to treat dermatological conditions and/or to provide cosmesis. Exemplary applications in the dermatological field include prevention or treatment of scarring of the dermis including hypertrophic scarring, dermal fibroproliferative lesions, and benign fibrous tumors such as keloids. In some embodiments, electron beam radiation may be used to treat or prevent scar formation resulting from breast cancer surgical procedures or reduce the severity of scar formation in emergency room procedures. System 10 also may be used to selectively target and disable cancer tissue relative to surrounding healthy tissue.

Advantageously, system 10 also may be useful to carry out therapies referred to as “FLASH” treatments. The so-called FLASH treatments use atypically high electron beam dose rates for atypically brief time duration(s) in one or more fractions, often only a single fraction. FLASH treatments have shown the ability of high energy electron beam energy delivered for brief dose intervals to selectively target and disable cancer tissue with minimal harm if any to surrounding healthy tissue. In particular, researchers have discovered that delivering higher dose rates of 50 Gy/s and higher, even up to 1000 Gy/s, or even up to 2000 Gy/s, vastly reduces healthy tissue toxicity while preserving anti-tumor activity.

FLASH techniques used in electron beam therapy by system 10 may use electron beam energies such as an energy of 4 MeV or higher, even 6 MeV or higher, even 12 MeV or higher such as up to 20 MeV, or even up to 50 MeV, or even up to 100 MeV. Flash techniques may deliver a total electron beam dose in a single treatment or single fraction such as a dose of at least 5 Gy, or even at least 10 Gy, or even at least 15 Gy such as up to 100 Gy. Flash techniques may deliver an electron beam dose in a relatively brief interval such as a treatment in the range from 0.01 milliseconds to 500 milliseconds, or even 0.1 milliseconds to 100 milliseconds.

In contrast to FLASH radiotherapy, the operating ranges of about 12 MeV or less, or even 6 MeV or less, generally are associated with lower levels electron beam energy in the field of electron beam therapy. Such energies, particularly those of about 4 MeV or less, are potentially more useful for shallow treatments, e.g., those in which the penetration depth (discussed further below) of the electron beam is in the range from about a fraction of 1 mm to several cm. For example, in illustrative embodiments involving therapy with limited penetration depth, system 10 may implement irradiation to depths in the range from is 0.5 mm or less to about 4 cm, preferably 1 mm to about 3 cm, more preferably 1 mm to about 1 cm. In preferred modes of practice, the therapeutic penetration depth is limited to about 1.5 cm or less. Undue bremsstrahlung production can be avoided with careful attention to avoid unnecessary objects in the path of the electron beam. Certain objects are beneficially presented to the electron beam, such as scattering foils, windows, absorbers (described further below), sensors, ion chambers and the like.

Consequently, as compared to FLASH radiotherapy, other modes of practice may use lesser energy, dose rates, and or doses to be delivered in one or more fractions for suitable time periods. For example, for some therapies, the electron beam energy delivered to the target site 12 is within a range from 0.1 MeV to 12 MeV, preferably 0.2 MeV to 6 MeV, more preferably 0.3 MeV to 4 MeV, and even more preferably 0.5 MeV to 2 MeV. In some modes of practice, an operation range from 1 MeV to 2 MeV would be desirable. In such embodiments, the electron beam systems provide irradiation doses of up to about 20 Gy, such as up to about 15 Gy, up to about 10 Gy, up to about 5 Gy, or up to about 2 Gy. In such embodiments, the electron beam systems provide radiation to the target site 12 at a rate of at least about 0.2 Gy/min, at least about 1 Gy/min at least about 2 Gy/min, at least about 5 Gy/min, or at least about 10 Gy/min. In such embodiments, the electron beam energy may be delivered to the target site 12 for a time period in the range from 0.01 milliseconds to 5 minutes, or even 0.1 seconds to 3 minutes.

For purposes of illustration, FIG. 1 shows system 10 being used to irradiate incised tissue proximal to a surgical incision 24 after wound closure in order to help reduce or prevent undue formation of scar tissue that otherwise could result as the incision subsequently heals.

Electron beam radiation system 10 of FIG. 1 generally includes an electron beam generation unit 26 that emits a linearly accelerated, straight through electron beam 16. Using feedback control techniques as described in U.S. Pat. No. 10,485,993, system 10 emits electron beam 16 with high stability and precision to achieve one or more desired penetration depth settings within a broad operating range. The feedback principles described in U.S. Pat. No. 10,485,993 allow the beam penetration depth, beam energy, dose, and/or dose rate to be rapidly adjusted and controlled in continuous or very small increments within the corresponding operating ranges. Being able to adjust these characteristics continuously or in small increments provides tremendous flexibility to tailor dose, energy, dose rate, and/or penetration depth to particular patient needs. This is a significant advantage over conventional machines that have only a limited number of energy settings and/or provide beams with less stability that are subject to coarser setting adjustments.

Penetration depth of an electron beam treatment means the R₈₀ penetration depth as determined in water according to the protocol described in Peter R. Almond et. al, “AAPM's TG-51 protocol for clinical reference dosimetry of high-energy photon and electron beams,” Med. Phys. 26 (9), September 1999, pp. 1847-1870 (referred to in the industry as the AAPM TG51 report). Note that while the protocol focuses on electron beams with mean incident energies in the range from 5 MeV to 50 MeV, the same protocol is applicable for lower or higher energies that optionally may be used in the practice of the present invention. Additionally, the report provides a protocol to determine the R50 penetration depth. This is the depth in water at which the absorbed dose falls to 50% of the maximum dose. The same depth-dose data resulting from this protocol also provides the R₈₀ penetration depth, which is the penetration of an electron beam dose into a water phantom at which the dose drops to 80% of the maximum dose. The depth of dose maximum is referred to as Dmax. Beam and dosimetry calibration for evaluation of machine settings with respect to determining R₈₀ penetration depth in the practice of the present invention are defined in water using a 5 cm diameter, circular, 30 cm long zero degree tip angle applicator at a 50 cm source to skin distance (SSD). The output for a specific energy is measured at Dmax.

For example, if this test shows that a particular machine configuration yields an R₈₀ penetration depth of 2 cm, that configuration is deemed to provide that R₈₀ penetration depth at the target site 12. The machine may be calibrated or otherwise evaluated to determine a plurality of machine configurations to correspond to a corresponding plurality of penetration depths. At the time of a procedure, the care provider selects a particular penetration depth suitable for the procedure. The machine is set to the corresponding configuration. The procedure is then performed using principles of the present invention to deliver a stable and precise electron beam as the procedure is carried out.

Electron beam energy and penetration depth are strongly correlated. See B. Grosswendt, “Determination of Electron Depth-Dose Curves for Water, ICRU Tissue, and PMMA and Their Application to Radiation Protection Dosimetry,” Radiat Prot Dosimetry (1994) 54 (2): 85-97. Depending on the embodiment, this relationship can be linear or nonlinear. Generally, higher penetration depth results from using electron beams with higher energy.

Still referring to FIG. 1, system 10 includes feedback control system 28 configured to permit controlling and adjusting the penetration depth, electron beam energy, electron beam dose, and/or electron beam dose rate provided by electron beam 16 with precision and stability using feedback strategies such as those described in U.S. Pat. No. 10,485,993. As shown in FIG. 1, control system includes at least one monitoring sensor that is used to detect at least two different characteristics of the electron beam 16. Monitoring in this embodiment includes at least two sensors in the form of first sensor 31 and a separate second sensor 34. In other embodiments, more sensors may be included. Alternatively, multiple sensor capabilities may be incorporated into a single sensor component. First sensor 31 measures a first characteristic (Si) of the electron beam 16. First sensor 31 sends a corresponding first sensor signal 32 to controller 38. Signal 32 corresponds to the value of the characteristic s1 measured by first sensor 31. Second sensor 34 measures a second characteristic s2 of the electron beam 16. Second sensor 34 sends a corresponding second sensor signal 36 to controller 38

Controller 38 uses the sensed information in order to implement feedback control in one or more aspects of unit 26. For example using strategies described in U.S. Pat. No. 10,485,993, controller 38 may use the sensed information to derive an analog characteristic, A, of electron beam energy from the detected characteristics s1 and s2 presented by the signals 32 and 36. The result is that measuring at least two different characteristics of the beam and using those to derive the analog characteristic allows characteristics of the electron beam 16, such as energy, dose, dose rate, penetration depth, and/or the like, to be easily controlled by control system 28 with high precision.

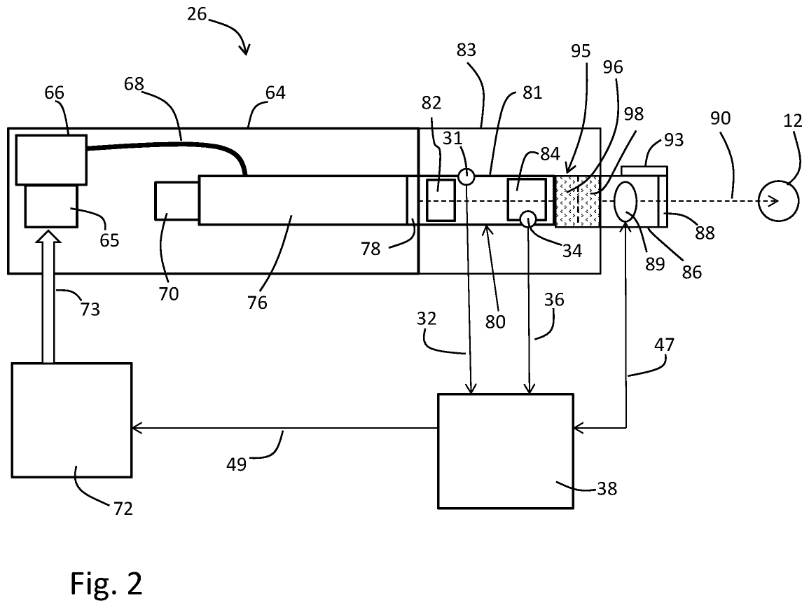

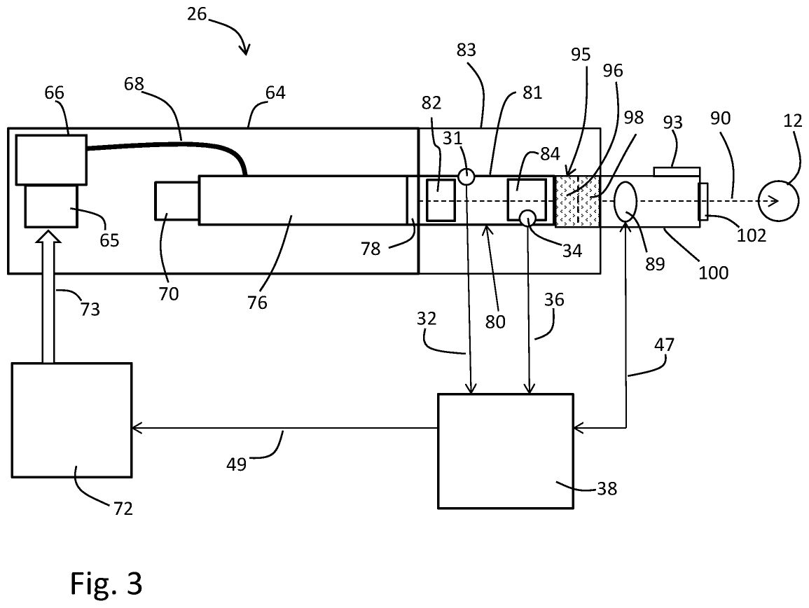

Controller 38 can use the control signal 40 in different ways to implement such feedback control. As one example, control signal 40 can be used to shut off the electron beam pursuant to an interlock protocol. As another example, control signal 40 can be used to adjust power source(s) that generate the electron beam in order to tune electron beam energy as desired. In some embodiments, such power-based control can be implemented by feedback control of the microwave source 66 (See FIG. 2 or 3) and/or the electron source 70 (See FIG. 2 or 3). Using the feedback control strategies, modulator or magnetron-based feedback (e.g., feedback to regulate modulator output voltage or magnetron frequency) allows adjusting electron beam energy in steps or continuously over the desired operating range, e.g., 0.1 MeV to 12 MeV in some embodiments, or even 6 MeV up to 20 MeV, or even up to 50 MeV, or even up to 100 MeV in other illustrative embodiments.

As another example, the modulator output voltage can be regulated to affect current supplied to the magnetron and the microwave power. The magnetron power may be regulated, which impacts the amount of power delivered to the accelerator 86 (FIGS. 2 and 3). In addition to these strategies or as an alternative to these strategies, feedback control strategies may be used with respect to other system features that are used to establish the electron beam, including gun voltage or the like. The gun voltage can be regulated to impact the launch velocity of electrons, phasing, capture, and energy spectrum.

As another approach to implement feedback control, control signal 40 can be used to adjust the settings of one or more physical system components, e.g., one or more electron beam absorbers, whose selected position setting can be used to modulate the electron beam energy. One such adjustable component is an electron beam absorber of variable thickness that can be adjusted to present different thicknesses, and hence different absorptions, to the electron beam 16. Such absorber-based control may be accomplished with single absorbing plates providing a range of selectable thicknesses, a variable thickness ribbon, or a rotating body whose degree of rotation presents variable thickness absorption to the electron beam. Using the feedback control strategies of the present invention, absorber-based feedback allows adjusting electron beam energy in steps or continuously over the desired operating range.

When using any absorber(s) to help tune the electron beam 16, control system 28 desirably includes monitors (not shown) that confirm that an absorber is in the correct installed position. If the monitors provide a signal indicating that the position is incorrect, an interlock protocol is triggered that prevents the electron beam from being turned on. Similarly, in those embodiments in which system 10 includes a plurality of absorbers with different thicknesses, a particular absorber or combination of absorbers is the proper absorber selection for carrying out a particular treatment at a desired penetration depth. Accordingly, control system 28 desirably contains monitors that check if the installed absorber matches the machine settings for the particular treatment. If the improper absorber is installed for the selected procedure, an interlock protocol is triggered that prevents the beam from turning on. As a further safety function, a particular treatment will usually involve delivery of a particular radiation dose. Control system 28 desirably monitors the delivered dose in real-time and initiates an interlock protocol to turn off the electron beam to avoid overdose.

Some embodiments of the present invention combine both power-based and absorption-based feedback control of the electron beam energy, dose, dose rate, and/or hence penetration depth.

Exemplary features of one embodiment of a suitable electron beam generation unit 26 useful in system 10 are shown schematically in FIG. 2. Unit 26 according to FIG. 2 incorporates an advanced applicator coupling system 95 in accordance with the present invention.

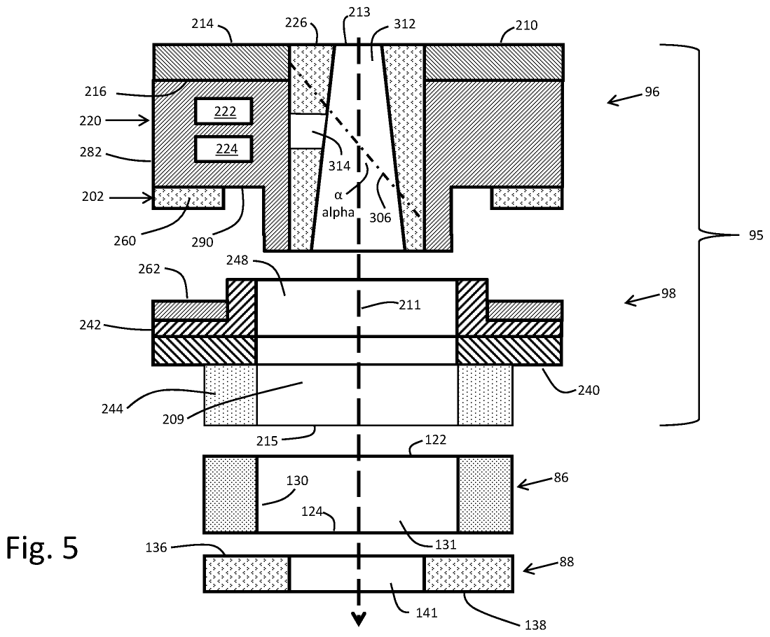

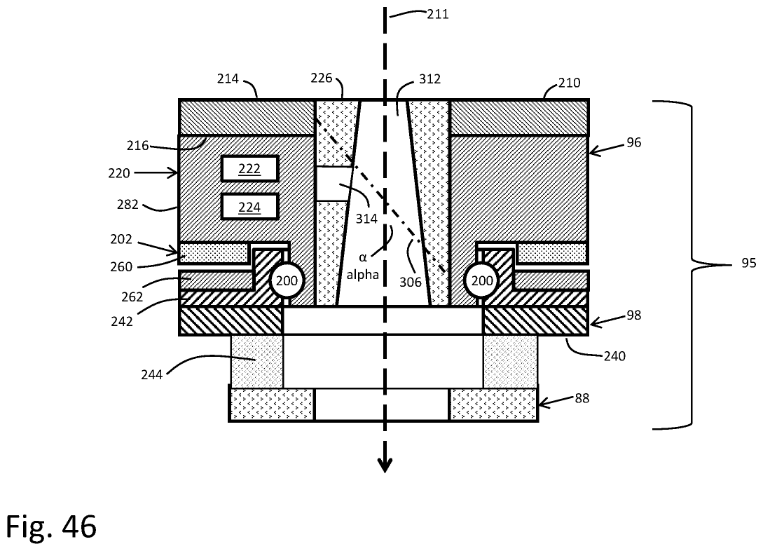

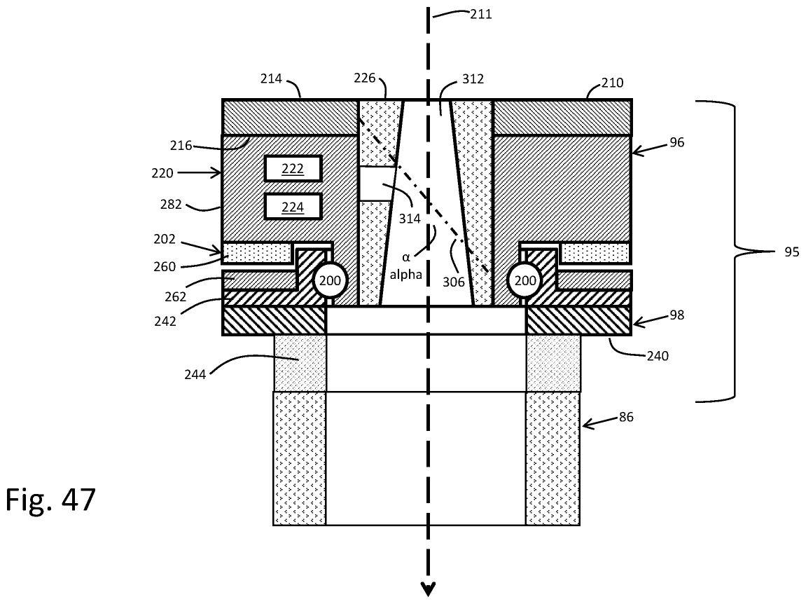

As seen in FIG. 2, electron beam generation unit 26 generally includes a first housing 64 that contains a modulator 65, microwave source 66, a microwave network 68, an electron source 70, and a linear accelerator 76. A second housing 83 contains a collimator 80. Using features of the present invention, the rotary coupling system 95 helps to rotatably mount on or more field defining members to be incorporated into unit 26. By way of example, system 10 is illustrated with a first field defining member in the form of an applicator 86 and a second field-defining member in the form of shield 88 integrated into the unit 26. Coupling system 95 generally incorporates a first sub-assembly 96 and a second sub-assembly 98, wherein the first sub-assembly 96 and second sub-assembly 98 are rotatably coupled to each other. The rotational coupling allows relative rotation between the two sub-assemblies 96 and 98 about an axis of rotation 211 (see, e.g., FIG. 5 and discussion below) that is parallel to, and desirably co-linear and coincident with, the central axis of the linear electron beam path 90. The coupling system 95 also incorporates automated distance detection, automated illumination functionality, and other functionality to be described further below. FIG. 46 below describes an embodiment in which shield 88 is attached to the rotary coupling system 95 to help shape the electron beam field, while the applicator 86 is not used. FIG. 47 below describes an embodiment in which applicator 86 is attached to the rotary coupling system 95 to help shape the electron beam field, while the shield 88 is not used.

An external power supply 72 supplies power to the modulator 65 via power cable 73. Power supply 72 and power cable 73 as an option may be included inside housing 64 along with other components. Controller 38 may be in communication with power supply 72 by communication pathway 49. An exit window 78 is provided at the interface between linear accelerator 76 and collimator 80. Scattering foil system 82 and ion chamber 84 are housed in collimator 80. Unit 26 generates an electron beam, which is aimed along substantially linear electron beam path 90 from accelerator 76 straight through applicator 86 to the target site 12 (also shown in FIG. 1). An optional field-defining shield 88 is placed at the exit of the applicator 86. A first sensor 31 is deployed with respect to collimator 80 for use in the feedback control strategies such as those described in U.S. Pat. No. 10,485,993. In such embodiments, ion chamber 84 among other functions also may serve as a second sensor 34 in such feedback control strategies.

Electron beam generation unit 26 as shown in FIG. 2 is the type that uses linear acceleration techniques to boost electron beam energy to desired levels. The use of linear accelerator structures to generate electron beams for therapeutic uses is well known. Additionally, electron beam generation unit 26 is a “straight through” type of system. As known in the art, a straight through system aims an electron beam at a target site along a generally linear path from the exit window 78 of the linear accelerator 76 straight through to the target site 12. This helps to ensure use of much of the beam current produced. Bending systems, in contrast, waste greater proportions of the beam current through absorption in bending magnet slits. Wastage of beam current in bending systems generally produces substantially greater background radiation per unit of dose delivered. A linear, straight-through beam line minimizes such beam loss and better optimizes dose per unit current to the target site. This means that the linear systems need less shielding. Straight through systems, therefore, tend to be smaller, more lightweight, and more compact than alternative systems that use heavy magnets and heavy shielding to aim electron beams on bent paths to a target site. An additional advantage of a straight through system is that energy may be varied quickly as there is no eddy current diffusion time limit or hysteresis as with bent beam systems. This makes linear, straight through systems more suitable for intraoperative procedures.

One example of such a system suitable for intraoperative procedures is described in U.S. Pat. No. 8,269,197 assigned to IntraOp Medical Corporation. Another example of such a system suitable for intraoperative procedures is the electron beam machine commercially available from IntraOp Medical Corporation under the trade designation MOBETRON. Generally, linear, straight through systems such as these are a result of engineering a compact linear accelerator that can fit when vertical under ceiling heights common to many procedure sites such as treatment rooms or surgery rooms. These compact systems avoid complex bending systems that tend to generate spurious background radiation that necessitates massive shielding.

Still referring to FIG. 2, modulator 65 receives power from the power output of power supply 72 via cable 73. Power supply 72 may be any suitable source of electricity. Power supply 72, as an option, may be a component of a continuous source of electricity from a power utility. Alternatively, power supply 72 may be battery powered, permitting untethered operation of electron beam generation unit 26. Modulator 65 accepts the power from power supply 72 (which may be line power, battery power or any suitable power source), and converts it to short pulses of high voltage that it applies to the microwave source 66. Microwave source 66 converts the voltage into microwave or RF energy.

Examples of suitable microwave sources for use as microwave source 66 include a magnetron or a klystron to power linear accelerator 76. A magnetron is more preferred as being less expensive and simpler to incorporate into system 10.

Many suitable embodiments of a magnetron operate using X-band, S-band, or C-band frequencies. X-band devices are more preferred, as other embodiments of unit 26 tend to be heavier when using S or C band devices. X-band frequency technology also tends to minimize the diameter, and hence the weight, of the accelerator structure. One illustrative example of a suitable magnetron operating at X-band frequencies is the Model L-6170-03 sold by L3 Electron Devices. This magnetron is capable of operating at a peak power of about 2.0 megawatts and 200 watts of average power.

Microwave network 68 conveys the microwave or RF power from the microwave source 66 to the linear accelerator 76. The microwave network 68 often typically includes a waveguide (not shown), circulator (not shown), a load (not shown), and an automatic frequency control system (not shown). The use of these components in an accelerator system is well known to those skilled in the art and has been described in the patent literature. See, e.g., U.S. Pat. No. 3,820,035. Briefly, microwaves from the RF source passes through the circulator before entering the accelerator guide to protect the RF source from reflected power from the accelerator 76. Instead, the power not absorbed in the accelerator 76 is reflected back into the circulator and shunted into a water-cooled or air-cooled dummy load. In the preferred embodiment, air-cooling is preferred as air cooling reduces weight and minimizes servicing issues. An AFC circuit is used to keep the resonant circuit tuned to the microwave frequency. Air cooling works in the practice of the present invention because magnetron average power, e.g., 200W in an illustrative embodiment, is relatively low for electron beams. In contrast, x-ray machines typically involve average power in the range from 1 kW to 3 kW. The ability to use air cooling with electron beams is one factor that helps preferred electron beam machines of the present invention to be so compact and lightweight.

Microwave or RF power may be injected into the accelerator 76 through a fixed waveguide if the microwave source 66 (e.g. a magnetron) is mounted on a rigid assembly (not shown) with the linear accelerator 76. Alternatively, a flexible waveguide may be used in the microwave network 68. As one option for implementing the feedback principles of the present invention, microwave or RF power supplied to the linear accelerator 76 through microwave network 68 may be modulated in the case of a magnetron by varying the pulsed high voltage supplied to the magnetron from power supply 72. Modulating the voltage of the power supply 72 in this manner allows the energy level, dose, dose rate, and/or penetration depth of the electron beam 16 to be controlled and adjusted to many different desired settings with excellent precision using the feedback strategies of the present invention. For a klystron, the same approach may be used. Alternatively, the input microwave power to the klystron may be varied.

In parallel with microwave source 66 supplying microwave or RF energy to linear accelerator 76, electron source 70 supplies electrons to linear accelerator 76. Electron source 70 typically includes an electron gun and features that couple the gun to the linear accelerator 76. Many different embodiments of electron guns are known and would be suitable. For example, some embodiments use a diode-type or triode-type electron gun, with a high-voltage applied between cathode and anode. Many commercially available electron guns operate at voltage ranges between 10 kV to 17 kV, though electron guns operating at other voltages may, in some embodiments, also be used. The voltage often is either DC or pulsed. In the case of the triode-type gun, a lower grid voltage also is applied between the cathode and grid. The grid can disable or enable the beam, and the grid voltage may be varied continuously to inject more or less gun current. The grid voltage may optionally be controlled through a feedback system. A skilled worker in the field of linear accelerator engineering is able to understand and choose an appropriate gun design suitable for the linear accelerator 76 to be used.

One example of a commercially available electron gun suitable in the practice of the present invention has been sold by L3 Electron Devices (formerly Litton) under the product designation M592 Electron Gun. The injector cathode of this particular gun operates in some embodiments at 10 kV to 14 kV and has a very small diameter emitting surface. This design is intended to provide low emittance and good capture efficiency while maintaining low energy spread. Typical pulse widths for operation may be in the range from 0.5 to 6 microseconds.

The RF source is pulsed by a modulator 65. It is preferred that the modulator 65 be solid state based rather than tube based to reduce weight and improve portability. The pulse repetition frequency (PRF) may be selected from a wide range such as from about 1 to about 500 pulses per second, and the pulse width may be selected from a wide range such as from about 1 to 25 microseconds. Some treatments can occur at these frequency rates and pulse widths for a particular time duration, e.g., from 0.5 seconds to 3 or even more minutes in some treatments. Other treatments may proceed for a given number of pulses and optionally fractional pulses such as from 1 to 50 pulses. The combination of PRF and pulse width is one factor that impacts the dose rate of the emerging electron beam. For diode-gun systems, the gun likewise may be pulsed by the same modulator system, albeit with an intervening gun transformer to permit a step in voltage.

Linear accelerator 76 is configured to receive the microwave or RF power from the microwave network 68. Linear accelerator 76 also is configured to receive the electrons from the electron source 70. Linear accelerator 76 is coupled to the microwave network 68 and the electron source 70 in a manner effective to use the microwave or RF power to accelerate the electrons to provide electron beam 16 having an energy in the desired operating range.

A variety of different linear accelerator structures would be suitable in the practice of the present invention. For example, linear accelerator 76 may have a structure that implements any of a variety of different cavity coupling strategies. Examples of suitable structures include those that provide side cavity coupling, slot coupling, and center hole coupling. C. J. Karzmark, Craig S. Nunan and Eiji Tanabe, Medical Electron Accelerators (McGraw-Hill, New York, 1993). Linear accelerator 76 also may have a structure that implements a variety of different symmetry strategies. Examples of suitable structures include those that provide periodic, bi-periodic, or tri-periodic symmetry. Examples of suitable accelerator structures also may implement a range of standing wave or travelling wave strategies. Examples of suitable linear accelerators 76 also may be selected to operate with many different bands of microwave or RF power. Examples of suitable power bands include S-Band (2-4 GHz), C-Band (4-8 GHz), X-Band (8-12 GHz), and still higher frequencies. David H. Whittum, “Microwave Electron Linacs for Oncology,” Reviews of Accelerator Science and Technology, Vol. 2 (2009) 63-92. In some illustrative embodiments, the linear accelerator 76 uses a low profile structure design, incorporating on-axis bi-periodic cavities operated at X-band frequencies. U.S. Pat. No. 8,111,025 provides more details on charged particle accelerators, radiation sources, systems, and methods, Side-coupled X-band accelerators and on-axis and side-coupled S-band and C-band accelerators are other suitable examples.

The linear accelerator 76, its attached electron source 70, and one or more other components of electron beam generation unit 26 may be mounted inside housing 64 on a strongback (not shown) or other suitable support member. The linear accelerator 76 and electron source 70 may be encased in lead or other shielding material (not shown) as desired to minimize radiation leakage. The higher the resonant frequency of the accelerator guide, the smaller is the diameter of the structure. This results in a lighter-weight encasement to limit leakage radiation. An advantage of linear, straight through machines is that the shielding requirements are less severe than machines that using beam bending strategies. This allows straight-through electron beam radiation machines to be deployed for intraoperative procedures rather than being deployed in remote locations inside heavily shielded rooms.

During operation, the network 68, the linear accelerator 76 and the microwave source 66 experience heating. It is desirable to cool unit 26 (particularly the units 65, 66, the circulator and loads in 68, and 76) in order to dissipate this heat. A variety of strategies can be used to accomplish cooling. For example, accelerator 76 and microwave source 66 can be water-cooled as is well known. In addition, the practice of the present invention permits operation at low-duty cycle, for which air-cooling would be quite adequate. The ability to practice air cooling simplifies the construction of unit 26 and helps to make the unit 26 smaller and more compact. The result is that the corresponding system 10 (See FIG. 1) is easier to deploy and use in intraoperative procedures.

An exit window 78 at the beam outlet of linear accelerator 76 is to help maintain a vacuum within the accelerator. The window 78 should be strong enough to withstand the pressure difference between the accelerator vacuum and the ambient atmospheric pressure, e.g., a difference of about 15 psi in some instances, but should be thin enough to avoid excessive beam interception and/or bremsstrahlung production. Balancing these factors, the window 78 may be formed of titanium in many embodiments. Alternatively, beryllium or other metallic or composite materials also may be used.

The accelerated electron beam 16 exits the linear accelerator 76 through exit window 78 and next continues on a linear path through collimator assembly 80 that receives, broadens, and flattens the beam. To implement feedback strategies of the present invention, one or more sensors may be deployed in or around collimator 80 in order to detect two or more independent characteristics of the beam. In the illustrative embodiment of FIG. 2, sensor 31 functions as a first sensor, and ion chamber 84, among its other functions, functions as a second sensor 34. Sensor 31 schematically is shown to the side of collimator 80, and thus generally out of the beam path in this embodiment. Other deployments, including deployments in the beam path or other locations downstream from exit window 78 may be used, if desired. For example, toroid devices are generally annular in shape and can be deployed so that the beam is transmitted through the open central region of the toroid.

Collimator 80 can include a housing 81. Housing 81 may be constructed of materials that help contain bremsstrahlung radiation, or the collimator design itself could be sufficient to contain the bremsstrahlung radiation. Inside housing 81, scattering foil system 82 and ion chamber 84 are provided. Scattering foil system 82 serves multiple functions. For example, electron beam systems typically produce beams of small transverse dimension, on the order of 1 mm to 3 mm across, much smaller than typical treatment fields. Scattering foil system 82 helps to broaden the electron beam 16. The scattering foil system 82 also helps to flatten electron beam 16. In many modes of practice, the beam passes through the scattering foil system 82 to help in shaping of the isodose curves at the treatment plane at target site 12.

In illustrative modes of practice, scattering foil system 82 helps to enlarge the accelerated beam 16 from being several square millimeters in cross section to several square centimeters in cross section. Uniformity of dose across the treatment field is a desired goal to simplify dose planning for therapeutic applications. For example, collimator 80 with or without applicator 86 may function to provide a flat electron beam dose profile such that the coefficient of variation of the beam dose across the full width at half-maximum (FWHM) of the beam is less than ±50%, less than ±40%, less that ±30%, less than ±20%, less than ±10%, less than ±5%, less than ±2.5%, or less than ±1%. Those of skill in the art will recognize that the coefficient of variation of the electron beam energy across the FWHM may have any value within this range, for example, about ±5%. In some embodiments, the collimator may function to broaden the electron beam to field sizes that are 1 cm to 25 cm across.

A typical scattering foil system 82 includes at least one, even two or more, and even three or more scattering foils (not shown). The distance between the two or more foils can vary, depending on the energy range of the unit, the field size needed for the treatment application, and the geometry and materials of the mass elements in the treatment head. Generally, electron scattering foils may be designed using techniques such as empirical design iteration or Monte Carlo simulations. Other means of providing uniformity could rely on magnetic phenomena. For example, steering coils could be employed to raster the beam across a programmed area. Alternatively, a quadrupole magnet system could be used to modify the beam size at the target plane.

Ion chamber 84 serves multiple functions. In one aspect, ion chamber 84 monitors the radiation dose delivered by the system and radiation when the prescribed pre-set dose is delivered. The monitor features of ion chamber 84 may be segmented transversely to provide a reading of beam position in the transverse plane. This reading may be used in a conventional feedback control system to provide current to steering coils upstream, so as to steer the beam and continuously correct any beam offset or symmetry error. Advantageously, in the practice of the present invention, this reading may be used in an innovative feedback control system (described further below) used to control the electron beam energy, and hence penetration depth at the target site, with excellent precision. As another function, ion chamber 84 may be used to terminate the beam and limit the amount of radiation received at the target site if an issue with the electron beam is detected. For example, a loss of a scattering foil could result in delivery of an excessive dose. In this fashion, ion chamber 84 and associated electronics provide protective interlocks to shut down the beam under such circumstances.

The first sub-assembly 96 of coupling system 95 is attached to the exit end of collimator 80. In the meantime, applicator 86 is attached to the exit end of the second sub-assembly 98. Field defining shield 88 (also referred to as an “insert”) is attached to the exit end of the applicator 86. Because second sub-assembly 98 is rotatably coupled to the first sub-assembly 96, this means that applicator 86 and the attached shield 88 are able to rotate about axis 211 relative to the first sub-assembly 96 and, hence, collimator 80 and other upstream components of unit 26. Rotation is helpful to help ensure that an appropriate alignment for the field defining opening (e.g., the outlet of the shield 88) with the treatment site, e.g., tumor, scar, incision, etc., is achieved.

If the applicator is metallic and could come into contact with the target site 12, the applicator 86 desirably is electrically isolated from the upstream components (e.g., coupling system 95, collimator 80, etc.) of system 10. This can be accomplished in various ways such as by interposing an insulative coupling between applicator 86 and second sub-assembly 98 or between applicator 86 and patient 14, or by forming applicator from a material that is inherently insulating (e.g., polymethyl(meth)acrylate often referred to as PMMA, quartz, ceramic, or the like).

The accelerated and collimated electron beam is aimed at a target site 12 through applicator 86 and field defining shield 88. The applicator 86 and shield 88 are configured so that the electron beam continues on linear electron beam path 90 straight through to the target site 12. In many modes of practice, the applicator 86 and shield 88 further help to define the shape and flatness of the electron beam 16. Applicator 86 also makes it easier to aim the electron beam while minimizing the manipulation of, contact with, or disturbance of the patient 14 or target site 12. Furthermore, the use of applicator 86 and shield 88 helps to avoid stray radiation and minimizes the dose delivered to healthy tissue by confining the radiation field.

Applicator 86 and/or shield 88 optionally may include one or more other components to help further modify the electron beam characteristics. For example, energy reduction with low bremsstrahlung can be achieved by interspersing thin (0.5-1 mm) sheets of plastic or sheets made from low atomic number material into the applicator 86 and/or shield 88 in a slot provided to accept them. Materials with higher electron density also may be used and could be thinner for the same absorption. The applicator 86 and/or shield 88 could also incorporate element(s) to act as a secondary scattering component. These may be made from suitable shaped low atomic number materials that help to further scatter electrons within the volume of applicator 86 and/or shield 88. Examples of such materials, but by no means exclusive to these materials, include aluminum, carbon, and copper and combinations of these. These can be located in applicator 86 at positions determined by Monte Carlo calculations or empirically for the energy and field size needed for the application.

In some modes of practice, a transparent or partially transparent applicator 86 and/or shield 88 may be beneficial. For example, such an applicator design may allow easier viewing of the treatment site. Applicators and or shields fabricated at least in part from PMMA, quartz, or the like would permit such viewing.

Unit 26 may be positioned in any orientation or position with respect to the target site 12 regardless of patient orientation. In many modes of practice, the distance from the exit end of the applicator 86 (or the end of field defining shield 88, if present) to the surface of the target site 12 can vary from contact with the target site 12 to distances up to about 10 cm from the patient surface. The distance can be determined by any suitable measurement technique such as by either mechanical measurement or an electronic rangefinder. Advantageously, coupling system 95 includes functionality that allows distance to be determined automatically. In some embodiments, the system 10 and/or applicator 86 may be positioned manually to achieve any orientation or position relative to the target site 12. In some embodiments, system 10 and/or the applicator 86 may be positioned using one or more motor drives for automated control of orientation and position. For example, the applicator 86 could be placed by hand and held in place by a suitable support structure (not shown). Then the electron beam machine would be docked (i.e., aligned) to the applicator 86. The applicator 86 desirably is electrically isolated from other components of system 10, particularly in treatments in which the applicator contacts or is close to the patient 14.

The applicator 86 may have a variety of shapes, such as being shaped to produce circular, square, irregular, or rectangular fields on the target site. Some useful applicators include cylindrical pathways for the electron beam to traverse. Another example of an applicator design, called a scan horn, creates long narrow fields by having scattering elements within the applicator that scatter electrons preferentially along the length of the field. In some embodiments, the scan horn may be used to confine the irradiated area to a strip of from about 2 cm to about 10 cm in length, and about 0.2 cm to about 1 cm in width.

FIG. 2 shows how an absorber 89 may be mounted on applicator 86 in a manner effective to tune the electron beam to adjust electron beam energy, dose, dose rate, penetration depth, or the like. By having a library of absorbers 89 with fine, stepwise differences in electron beam absorption, different adjustments of the electron beam in fine increments can be delivered to treatment sites such as site 12. In the meantime, feedback strategies such as those described in U.S. Pat. No. 10,485,993 are used to stabilize the electron beam with high precision prior to tuning by the absorber 89. To change to another penetration depth setting, one or more different absorbers 89 are presented to the beam and/or the machine may be set to produce an electron beam with a different energy level that is presented to the one or more absorbers 89. The different absorbers 89 may be installed manually or via automation. U.S. Pat. No. 10,485,993 further describes how to use absorbers to help adjust an electron beam.

FIG. 2 shows absorber 89 mounted to applicator 86. The absorber 89 may be located in other positions and still provide effective tuning. Generally, the absorber 89 is deployed in the path 90 of the electron beam between the exit window 78 and the target site 12. Many suitable embodiments of absorber 89 are fabricated from one or more low Z materials above atomic number 4. Exemplary materials useful to form absorber 89 include carbon, aluminum, beryllium, and combinations of two or more of these. Higher Z materials could be used, but with the risk of generating undo amounts of Bremsstrahlung radiation. Controller 38 may be in communication with absorber(s) 89 via communication pathway 47.

FIG. 2 shows machine vision capability integrated with unit 26. In some embodiments, machine vision is achieved by mounting one or more endoscopes 93 onto applicator 86. Endoscope 93 allows real time video imaging of target site 12. Endoscope 93 or other machine vision capability is helpful to allow target site 12 to be viewed without obstruction by applicator 86, shield 88, or other components of system 10. As one advantage, endoscope 93 allows real time viewing of target site 12 as system 10 is set up and aimed at the target site 12. This can be helpful to make sure that system 10 is aimed properly at site 12 without undue misalignment or tilting. An operator can also view the captured image information to observe the site 12 during a treatment. This will allow the operator to capture image information to document the treatment. Also, the operator can observe to make sure that the patient 14 does not move out of the proper set up as a treatment proceeds. Endoscope 93 is very suitable for this, as endoscopes generally are flexible for easy mounting, capture high quality, real time images, and are inexpensive.

FIG. 3 shows an alternative configuration of unit 26 FIG. 2. Unit 26 of FIG. 3 is identical to unit 26 as shown in FIG. 2 except that a different applicator 100 and an alternative field defining shield 102 are used. In this illustration, applicator 100 is longer than applicator 86 (FIG. 2), while shield 102 is smaller and helps shape a more tightly defined electron beam field than shield 88 (FIG. 2). FIG. 3 shows the modular capabilities of unit 26 with respect to independently choose and use different applicators and/or shields to easily adapt to the needs of a variety of different electron beam treatments and circumstances. The applicators are modular in the sense that a library may include an inventory of two or more applicators, each of which is interchangeably mounted on the unit 26. Similarly, the shields are modular in the sense that a library may include an inventory of two or more shields, each of which is interchangeably mounted on the unit 26.

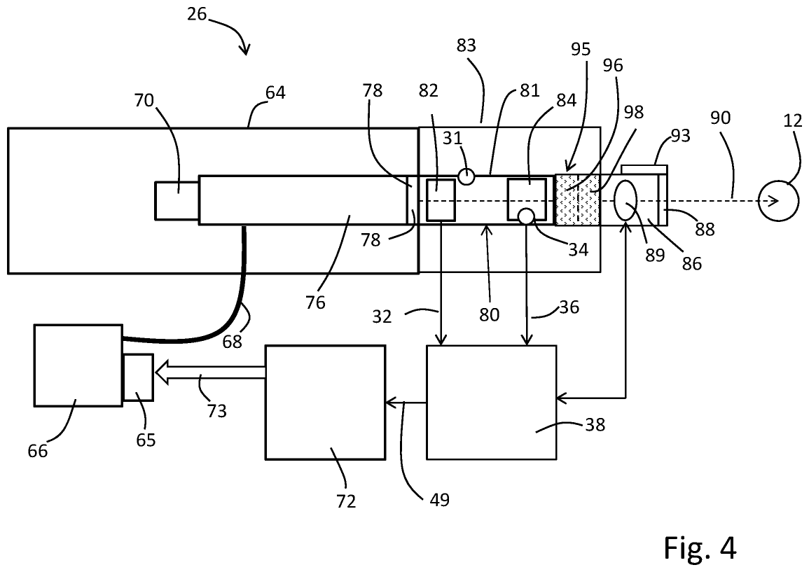

FIG. 4 shows another alternative embodiment of the electron beam generation unit 26. The unit 26 of FIG. 4 is identical to the system 10 of FIG. 2 except that the microwave source 66 and a portion of the microwave network 68 are external to housing 64. Rotational motion between the two ends of the network 68 can be practiced by incorporating one or more rotary joints into network 68 according to conventional practices.

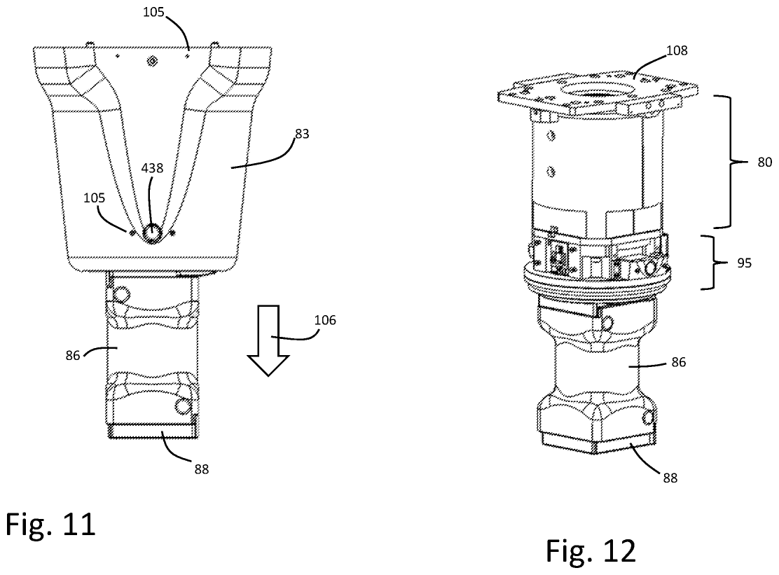

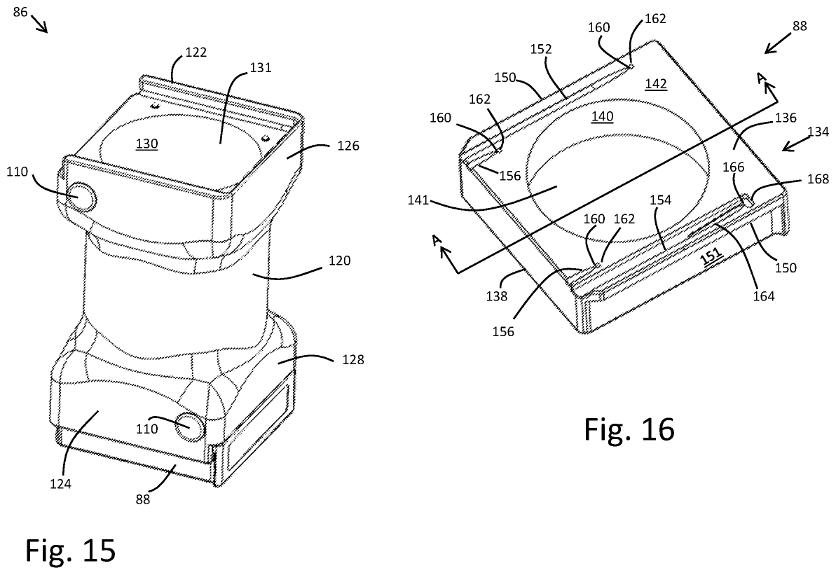

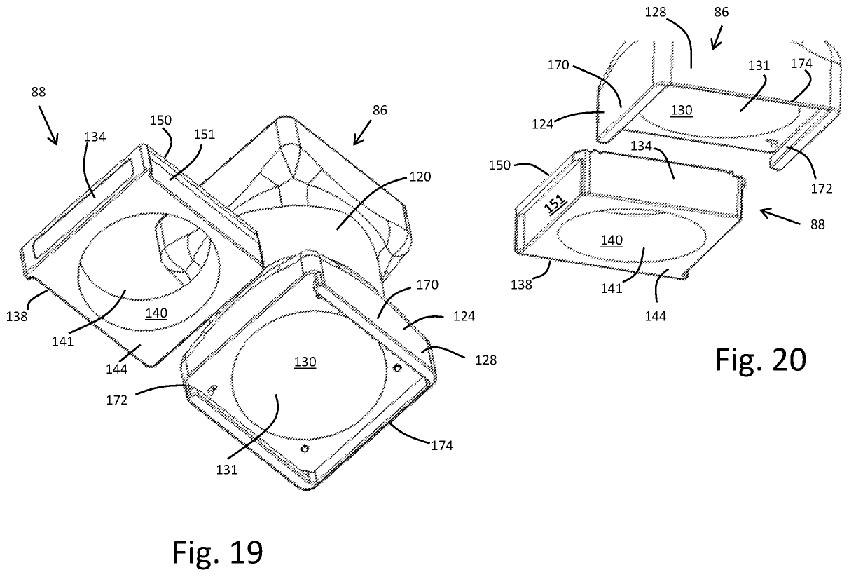

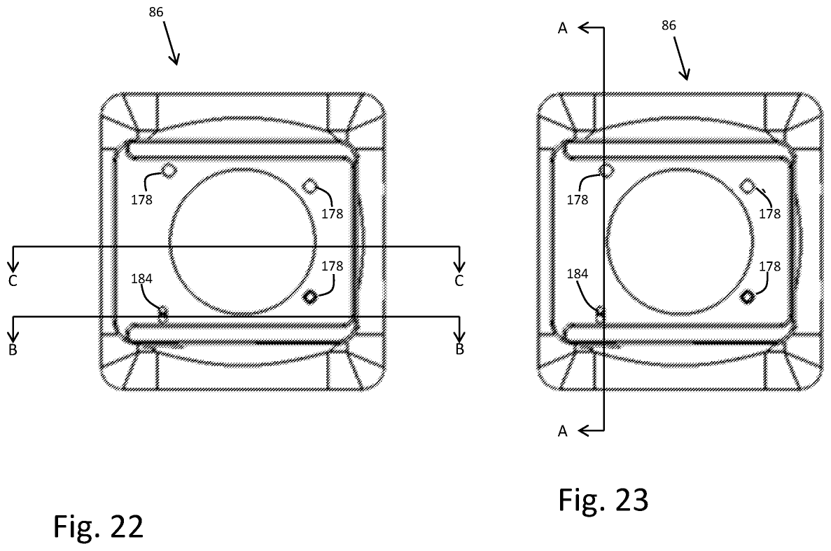

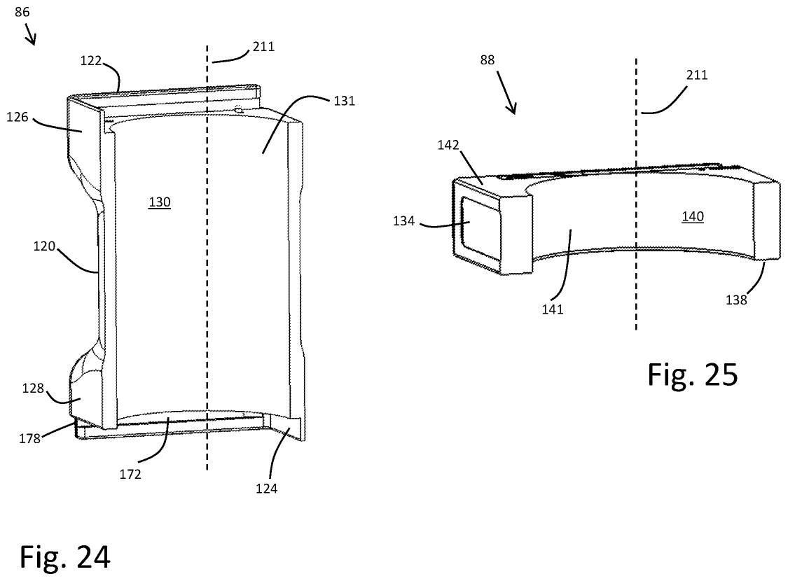

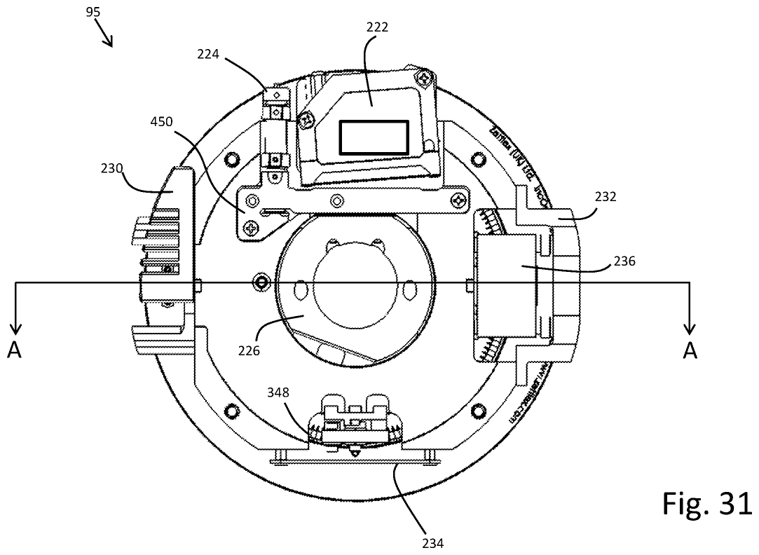

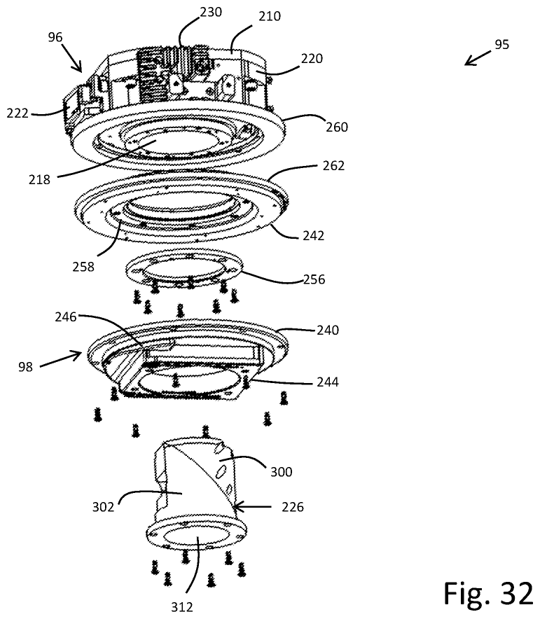

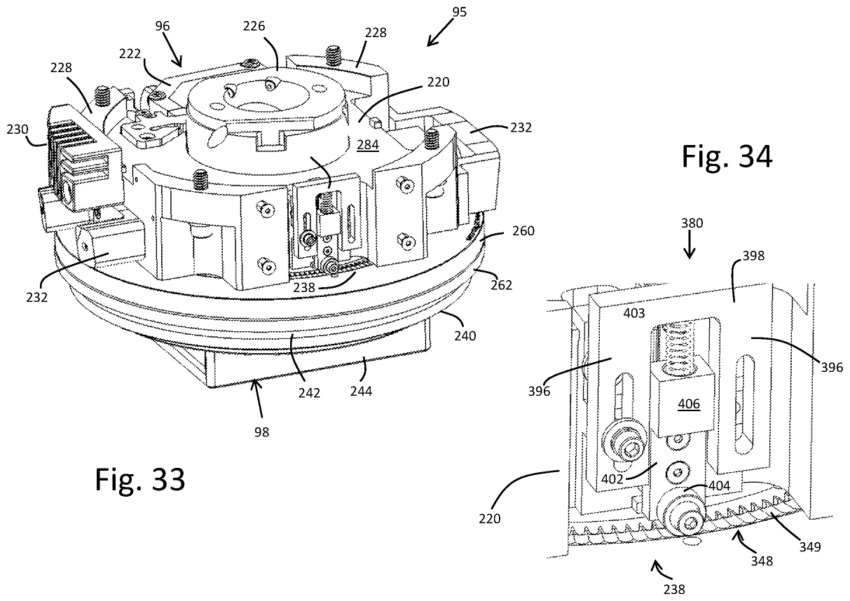

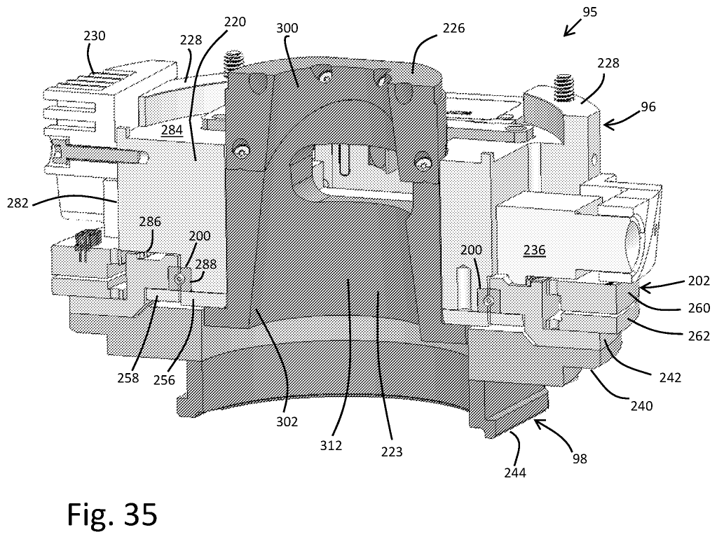

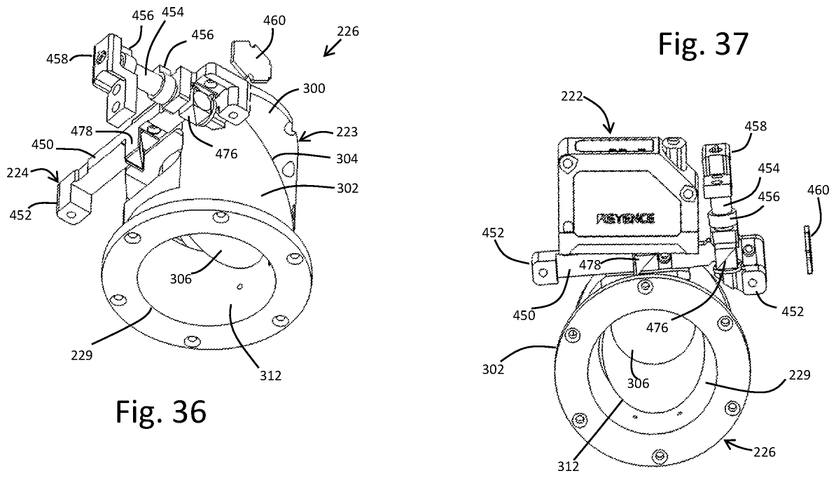

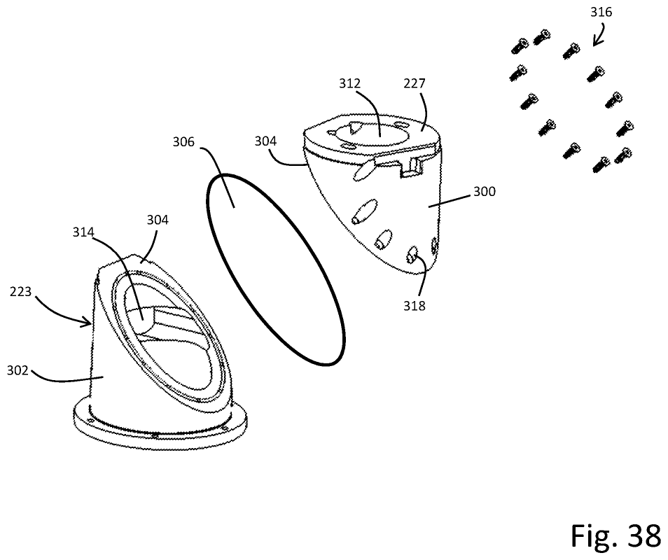

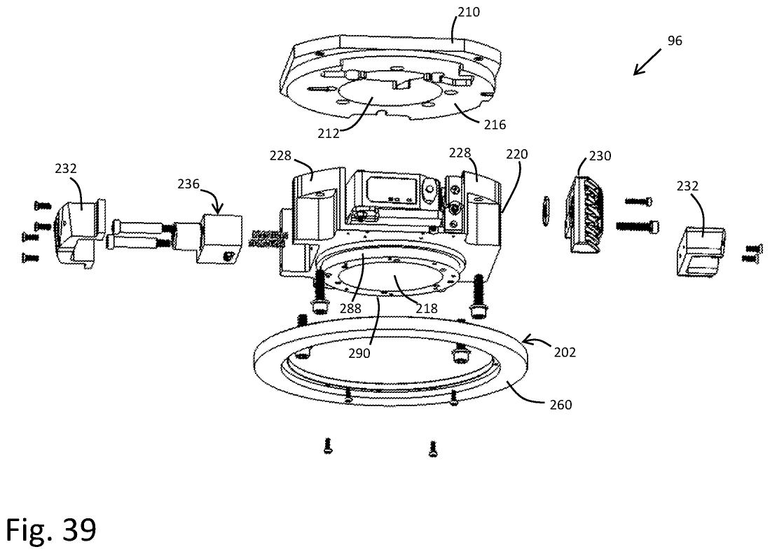

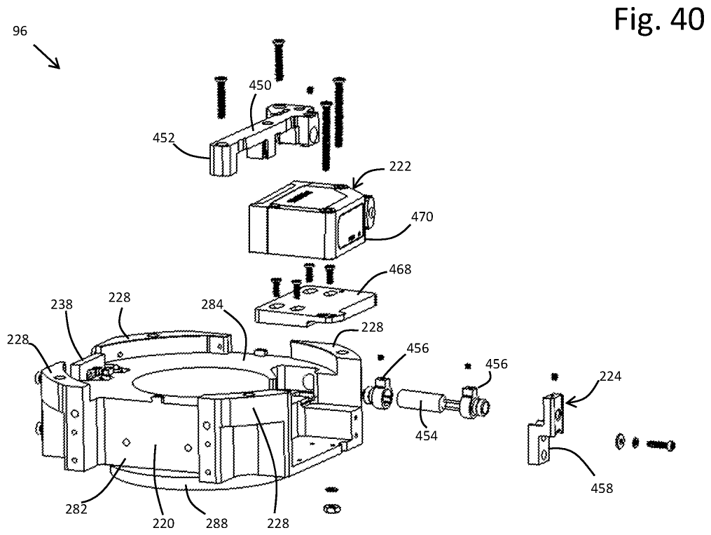

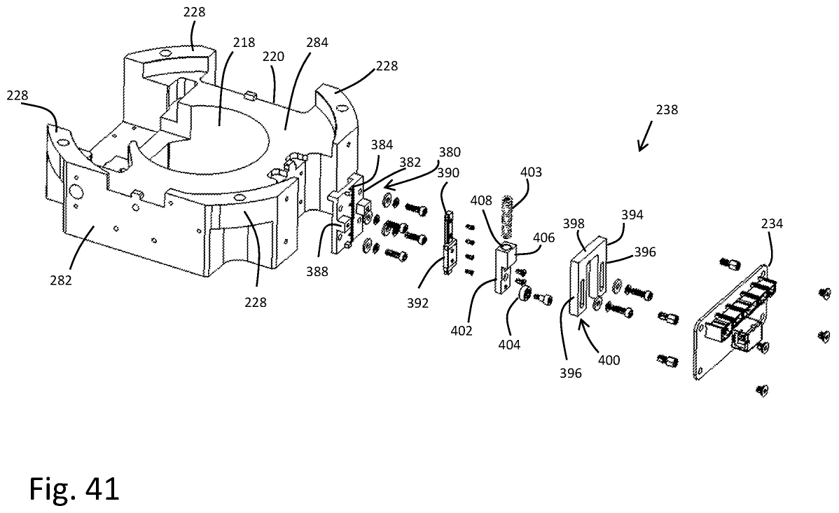

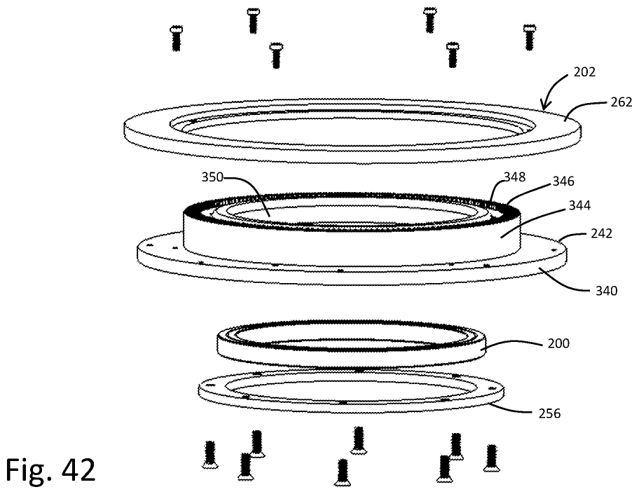

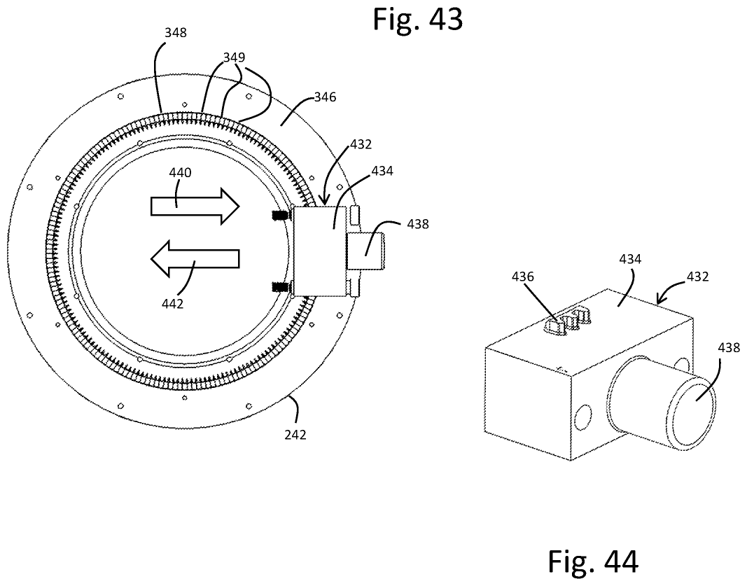

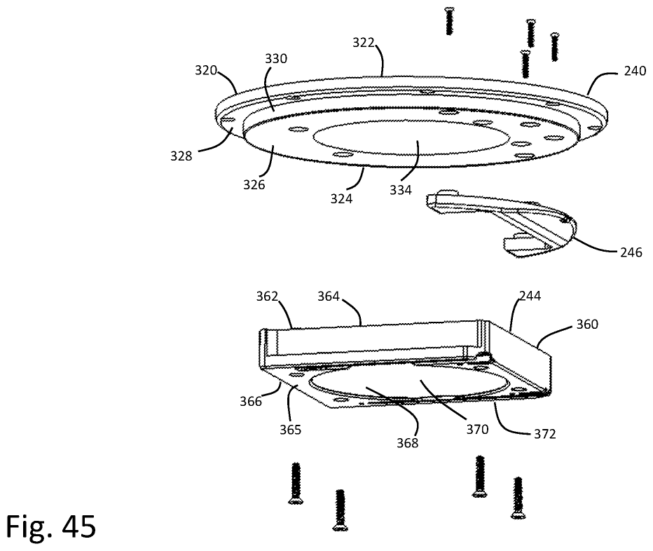

FIGS. 5 to 45 show the applicator 86, shield 88 and coupling system 95 of FIGS. 1 and 2 in more detail. Referring first to FIGS. 11 and 12, FIGS. 11 and 12 show how easily housing 83 is mounted and de-mounted from unit 26. FIG. 11 shows how housing 83 is mounted over the collimator 80 (not shown in FIG. 11 as being under housing 83) and the coupling system 95 (not shown in FIG. 11 as being under housing 83) using screws 105 to help hold housing 83 in place. The applicator 86 and shield 88 are accessible below the housing 83. To remove housing 83, the screws 105 are removed. Similar screws are on the other side of housing 83 as well. Removing the screws 105 releases the housing 83. This allows the housing to be removed from unit 26 in the direction shown by downward arrow 106. Note that button 438 is used for the separate function of releasing rotational locking functionality so that the applicator 86 can be rotated. FIG. 12 shows the uncovered unit 26 after housing 83 is removed. The collimator 80 and coupling system 95 are now exposed. A mounting plate 108 also is shown at the top of the collimator 80. Mounting plate 108 is used to attach collimator 80 to upstream components of unit 26.

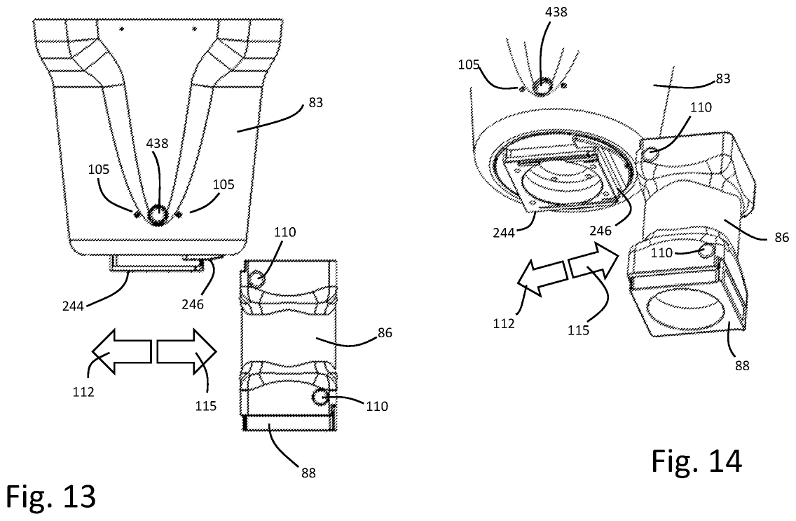

FIGS. 13 and 14 show how applicator 86 and an attached shield 88 are easily mounted and de-mounted from a mounting plate 244 on the lower end of the coupling system 95. To mount, applicator 86 and mounting plate 244 include complementary features that allow applicator 86 to be simply slid onto mounting plate 244 in the direction of arrow 112. Front plate 246 is provided in a different color than mounting plate 244 and housing 83 to help provide a visual guide to mount applicator 86 from the right direction. The leading face of mounting plate 246 has a shallow bevel in order to help guide applicator 86 onto mounting plate 244. At the time of mounting, shield 88 may already be attached to applicator 86. Alternatively, shield 88 may be mounted onto applicator 86 at a later time. Once mounted to mounting plate 244, because second, downstream sub-assembly 98 is rotatable with respect to the first, upstream sub-assembly 96 about axis 211 (see FIGS. 5-8), applicator 86 and shield 88 mounted to the second sub-assembly 98 are rotatable about the same axis 211 as well. Mounting plate 244 and applicator 86 include complementary mounting features (described further below) that help to mount and lock applicator 86 in place.

De-mounting of applicator 86 from mounting plate 244 is easy. Button 110 is pushed to release locking features described below. This allows applicator 86 to be slid off of mounting plate 244 in the direction of arrow 115. Similar, complementary mounting and de-mounting features (described further below) also are used to mount the shield 88 to the applicator 86.

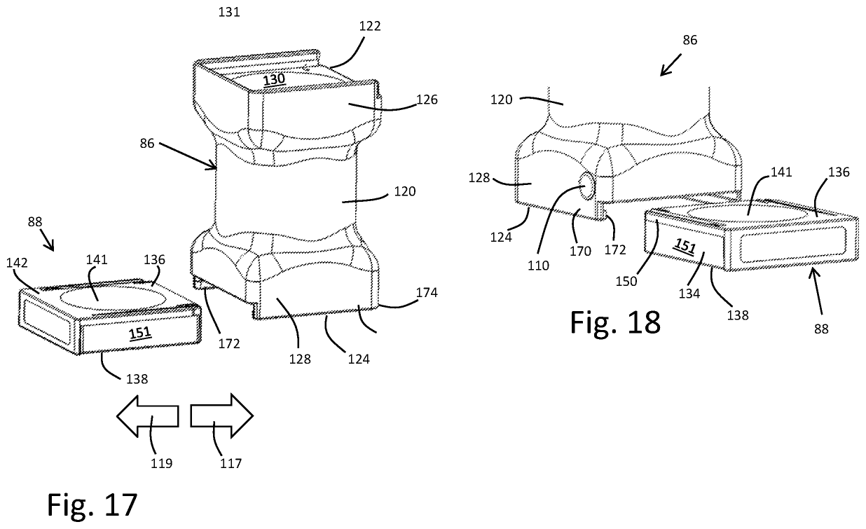

FIGS. 17 to 20 show how shield 88 is easily mounted and de-mounted from applicator 86. To mount, applicator 86 and shield 88 include complementary features that allow shield 88 to be simply slid onto applicator 86 in the direction of arrow 117. Shield 88 and applicator 86 include complementary mounting features (described further below) that help to mount and lock applicator 86 in place.

De-mounting of shield 88 from applicator 86 is easy. Button 110 is pushed to release locking features described below. This allows shield 88 to be slid off of applicator 86 in the direction of arrow 119. Similar, complementary mounting and de-mounting features (described further below) also are used to mount the applicator 86 to mounting plate 244.

FIGS. 5 to 7 and 11-28 show applicator 86 and shield 88 in more detail. Applicator 86 includes body 120 extending from a first inlet end 122 proximal to the coupling system 95 (FIG. 1) to a second, outlet end 124. Head 126 is at first inlet end 122. Head 126 includes mounting features (described below) used to mount applicator head 126 onto the outlet end of the mounting plate 244. Foot 128 is at second outlet end 124. Foot 128 includes mounting features (described below) used to mount applicator foot 128 to shield 88. Applicator 86 includes through aperture 131 defined at least in part by interior surface 130. Aperture 131 has a length that is centered about axis 211. Aperture 131 provides a pathway for the electron beam 16 (FIG. 1) to travel through aperture 131 from the inlet end 122 to the outlet end 124.

Field defining shield 88 (also referred to in the industry as an “insert”) has body 134 extending from a first inlet end 136 to a second, outlet end 138. Top face 142 is at inlet end 136. Lower face 144 is at outlet end 138. Shield 88 includes a through aperture 141 defined at least in part by interior wall 140. Aperture 141 has a length that is centered about axis 211. Aperture 141 provides a pathway for the electron beam 16 (FIG. 1) to travel through aperture 141 from the inlet end 136 to the outlet end 138.

FIGS. 13 to 28 show coupling features used to mount and de-mount the applicator 86 from the mounting plate 244 and the shield 88 from applicator 86. The same coupling features are used both respect to the head 126 and foot 128 of the applicator 86. For brevity, the features associated with mounting and de-mounting the applicator 86 and shield 88 at the foot 128 of applicator 86 are described, with the understanding that the features associated with the mounting plate 244 and head 126 of applicator 86 are of the same type.

First, mounting features on the top face 142 of shield 88 are described. Similar features are incorporated into mounting plate 244. Rails 150 extend along opposite sides 151 of shield 88 at the inlet end 136. The top face 142 includes long slot 152, a long wide slot 154, and short slots 156 extending along top face 142 generally parallel to rails 150. The ends of slots 152 and 156 include constrictions 160 defining terminal ends 162. Ramp 164 having backstop 166 is provided on one side proximal to the end of wide slot 154. Pocket 168 is formed behind backstop 166 on one side of wide slot 154.

Mounting features at the foot 128 of applicator 86 are now described. Foot 128 includes sidewalls 170 including slots or tracks 172. The tracks 172 are open at one end and terminate at backwall 174. Foot 128 includes a plurality of plungers 178 that are able to move up and down but are biased, such as by a spring, to be in a lowered position. The plungers 178 are deployed to ride in slots 152 and 156 of shield 88 when shield 88 is mounted to and held on foot 128. The plungers 178 are able to ride up over the constrictions 160 and become releasably held in the pockets 168. Pulling or pushing on shield 88 causes the plungers 178 to engage or be released from the pockets 168. Plungers 178 have tapered heads to facilitate this engaging and releasing function.

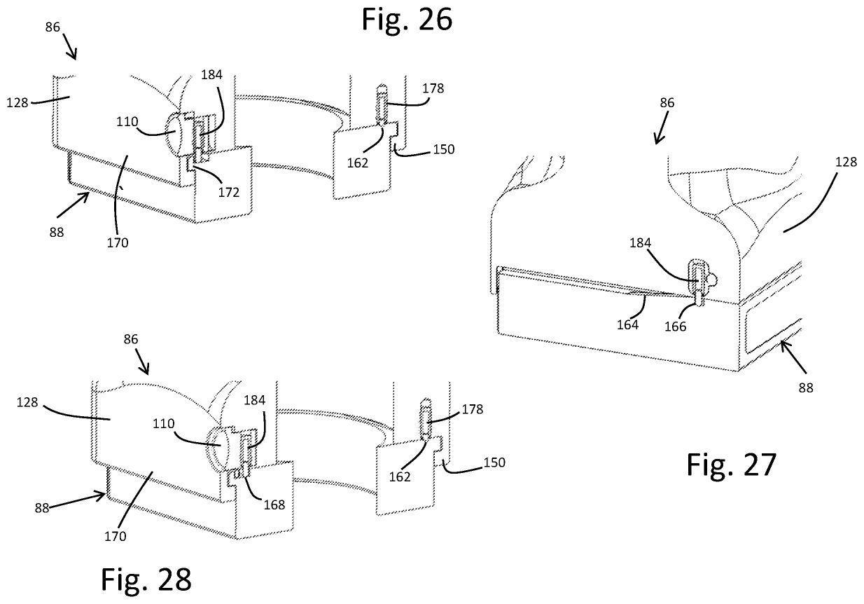

Releasable locking functionality is provided by button 110 and shiftable plunger 184. Button 110 engages shiftable plunger 184. Shiftable plunger 184 not only is able to move up and down in a similar spring-biased manner as plungers 178, but also plunger 184 has a side-to-side range of motion based on button actuation. When not actuated (FIGS. 26 and 27), button 110 tends to be in an un-pressed configuration in which plunger 184 is biased to be on the same side of track 154 as ramp 164. In this configuration, plunger 184 is able to ride up ramp 164 when shield 88 is inserted onto foot 128 and then is trapped behind ramp 164 when shield 88 is fully inserted onto applicator 86. This locks shield 88 onto applicator 86. When button 110 is pressed (FIG. 28), plunger 184 is pushed over to the other side of track 154 so as to be clear of ramp 164. This unblocks plunger 184, and hence unlocks shield 88, allowing shield 88 to be removed from applicator 86. Plunger 184 has a rounded head to facilitate this locking and unlocking functionality.

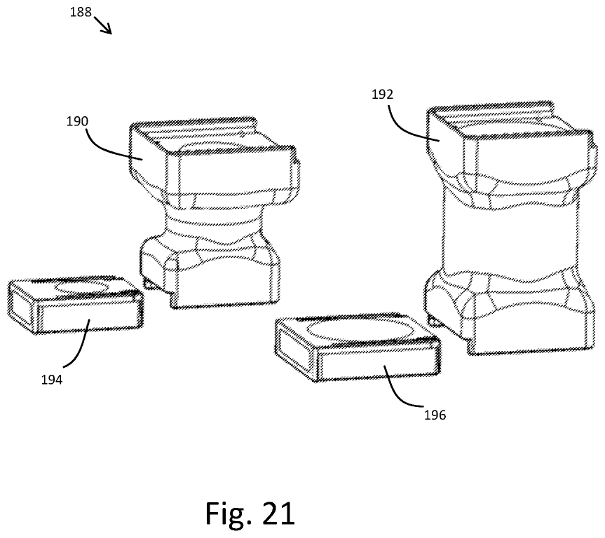

FIG. 21 schematically shows how system 10 of FIG. 1 may include a library 188 of absorbers and shields. Such a library may include two or more shields 194 and 196 and two or more applicators 190 and 192. Each type of applicator may be interchangeably attached to two or more different shields 194 and 196. In some modes of practice, different sized applicators 190 and 192 may be compatible with different sets of shields 194 and 196, respectively. The applicators 190 and 192 and shields 194 and 196 may differ in terms of a variety of characteristics such as material(s) of construction, length, diameter, geometry of the central aperture through which the ebeam travels, interior accessories, exterior accessories, and the like. The components of a library may include detection features so that system 10 can automatically detect which component is used and thereby provide custom interfaces or choices associated with the identified component.

For purposes of illustration, FIG. 21 shows library 188 including a small applicator 190 and a large applicator 192. One or more small shields 194 are associated with small applicator 190. One or more larger shields 196 are associated with the large absorber 192.

FIGS. 5-7, 12, and 29-45 provide an overview of the coupling system 95 and its main components. Coupling system 95 generally includes a first, upstream sub-assembly 96 that is rotatably coupled to a second, downstream sub-assembly 98. A rotary encoder 202 is incorporated into coupling system 95 so that the relative rotation between sub-assembly 96 and sub-assembly 98 can be automatically monitored and measured. System 95 includes a main central aperture 209 and a main central axis 211. Central aperture 209 provides a pathway for ebeam 16 (FIG. 1) to pass through from inlet 213 to outlet 215. Inlet 213 is coupled to upstream components of unit 26 (FIG. 1). Outlet 215 is coupled to applicator 86. Automated functionality (described below) for measuring distance to the target site 12 (FIG. 1) and automated functionality (described below) for aiming the ebeam 16 and illuminating the target site 12 are incorporated into the system 95.

First, upstream sub-assembly 96 generally includes an upper mounting plate 210 used to attach sub-assembly 96 to upstream components. Mounting plate 210 includes a central aperture centered about axis 211, an upper or upstream face 214, and a lower or downstream face 216. Mounting plate 210 is coupled to mounting bosses 228 on main body 220. Main body 220 includes a central aperture 218 that houses central core and mirror assembly 226. Central core and mirror assembly 226 in turn has central aperture 312 along central axis 211 through which the ebeam 16 (FIG. 1) travels.

Main body 220 incorporates many systems that provide several advantageous functions and capabilities. Distance detection system 222 and optical illumination system 224 are integrated with main body 220. Additionally, a rotary locking and release mechanism 236 and rotary indexing system 238 also are integrated with main body 220. Heat sink 230 is provided to help dissipate heat generated from the LED light source 460. A controller 234 is mounted to main body 220 as well.

A portion of the rotary encoder 202 is also mounted to main body 220. Rotary encoder 202 includes stator ring 260 and rotor ring 262. Stator ring 260 is mounted to main body 220, while rotor ring 262 is mounted to the second-subassembly 98. The rotary encoder 202 incorporates electronic capabilities so that the rotational position of stator ring 260 relative to the rotor ring 262 is easily monitored and measured. The result is that the relative rotation of the sub-assembly 96 relative to the sub-assembly 98 is easily and accurately monitored, such as to a fraction of a rotational degree if desired. In some embodiments, the rotary encoder 202 includes absolute encoder functionality so that the rotation position is known even if power is lost. Mounting features are used to help mount housing 83 (FIG. 11) onto coupling system 95. The main components and functions of first, upstream sub-assembly 96 are described in more detail below.

Lower, downstream sub-assembly 98 includes several main components as well. These include rotary base plate 240, rotor 242, mounting plate 244, and front plate 246. Rotor ring 262 of rotary encoder 202 is incorporated into sub-assembly 98 as well. Lower sub-assembly 98 includes central aperture 248 having central axis 211. The main components and functions of second, downstream sub-assembly 98 are described in more detail below.

Annular ring bearing 200 rotatably couples first, upstream sub-assembly 96 to second, downstream sub-assembly 98. This allows sub-assembly 96 to rotate relative to sub-assembly 98. In practice, sub-assembly 96 is attached to a larger assemblage of upstream components of unit 26 (FIG. 2), while second sub-assembly 98, the applicator 86, and shield 88 are rotatable on demand about axis 211. Ring bearing 200 includes inner race 250, outer race 252, and ball bearings 254. Inner race clamp 256 holds inner race 250 in place with respect to first sub-assembly 96. Outer race clamp 258 holds outer race 252 in place with respect to second sub-assembly 98.

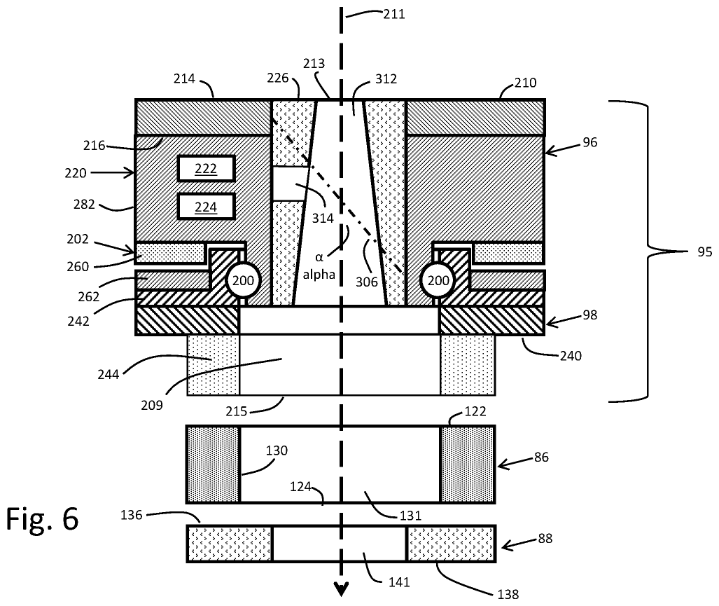

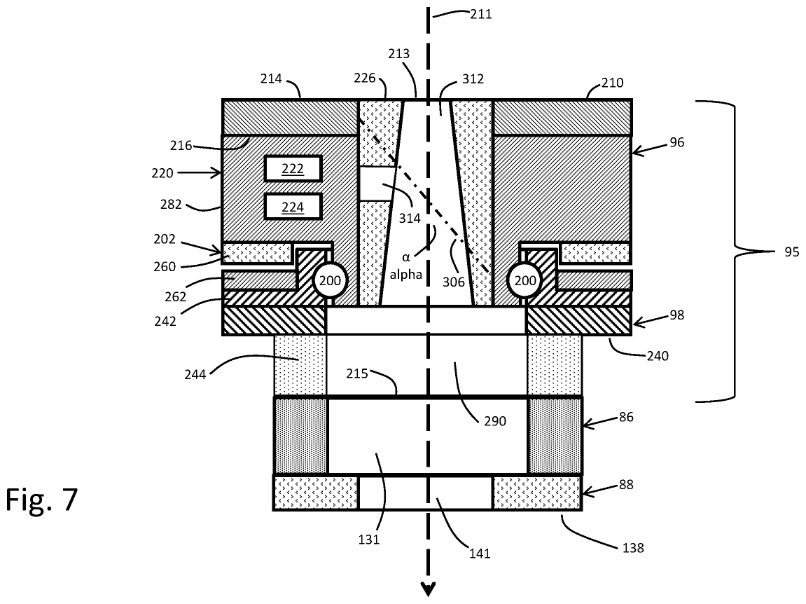

FIGS. 5 to 8 provide an overview of how the main components of the first sub-assembly 96, second sub-assembly 98, applicator 86, and shield 88 are assembled to provide the applicator 86 and attached shield 88 with rotational functionality. FIG. 5 shows the separate components 96, 98, 86, and 88 separately aligned on axis 211. In FIG. 6, the sub assemblies 96 and 98 are rotatably coupled together by ring bearing 200. This assembly provides the coupling system 95. In FIG. 7, the applicator 86 is attached to the lower sub-assembly 98, and the shield 88 is attached to the applicator 86.

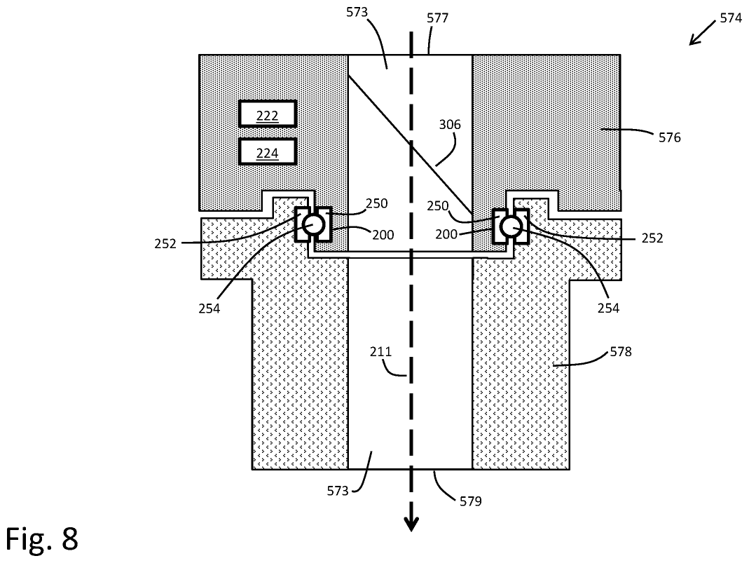

As shown in FIG. 8, the resultant assembly 574 can be viewed has having a first unit 576 rotatably coupled to a second unit 578. The first unit 576 corresponds to the first, upstream sub-assembly 96. The second unit 578 can be viewed as a singly assembly that corresponds to the assembled second, downstream sub-assembly 98, the applicator 86, and the shield 88. The assembly includes the main central aperture 573 having central axis 211 through which electron beam 16 (FIG. 1) passes from inlet 577 to outlet 579.

FIGS. 5-7, 29-33, 35-39, 40, 41 show the main body 220 in more detail. Main body 220 includes sidewall 282, top 284, shoulder 286, neck 288, and lower face 290. Mounting bosses 218 on the top 284 are used to attach the mounting plate 210. Central aperture having central axis 211 is provided to house the central core and mirror assembly 226.

FIGS. 5-7, 29-33, 35-38 show the central core and mirror assembly 226 in more detail. Central core and mirror assembly 226 has body 223 having a central aperture 312 extending along central axis 211. Central aperture 312 provides a pathway for ebeam 16 (FIG. 1) to pass from inlet 227 to outlet 229. Body 223 is provided by upper (upstream) member 300 and lower (downstream) member 302 that are joined at interface 304. Interface 304 provides clamping surfaces that clamp mirror 306 in place between member 300 and member 302. The interface is formed so that the mirror is held at a tilted angle relative to the central axis 211. The term “tilted” means that the mirror 306 is clamped so that its reflecting face(s) are non-orthogonal and non-parallel to central axis 211. Generally, as the mirror 306 is tilted relative to the central axis 211, one side of the mirror will have an acute angle alpha with respect to the axis 211. The angle alpha desirably is in a range from 10 degrees to 80 degrees, even 20 degrees to 70 degrees, or even 30 degrees to 60 degrees. In one embodiment, holding the mirror 306 at a tilted angle of 45 degrees would be suitable.

It can be seen that the mirror 306 is mounted at a tilted angle in the through aperture 312 of the central core and mirror assembly 226 that has a conical shape that progressively opens as the ebeam moves downstream through the assembly 226. At the same time, the assembly 226 is desirably formed from a polymer material that has ebeam absorbing characteristics. This helps to reduce stray radiation and x-ray production.

Mirror 306 advantageously is at least partially reflective to optical illumination (e.g., electromagnetic light include one or more wavelength portions in a range from ultraviolet light (e.g., as low as about 200 nm) to infrared light (e.g., as high as about 2000 nm). More desirably, mirror 306 is at least partially reflective to visible light such as one or more wavelength bands in a range from 430 nm to 750 nm. An advantage of a mirror face that is partially reflective to such light is that it allows distance detection and illumination components to be housed outside of central core and mirror assembly 226 where these can laterally transmit light generally radially inward toward the central axis 211. Mirror 306 redirects the light downward along axis 211 to accomplish illumination and distance detection operations as described further below.

Because mirror 306 is clamped within central aperture 312 in the ebeam path, it is desirable that mirror 306 is at least partially transparent to the ebeam while still also being partially reflective with respect to the optical illumination. A mirror configuration will be deemed to be partially transparent to ebeam radiation if any portion of the electron beam incident on the upstream face of the mirror is able to reach the target site 12 (FIG. 1). Even though an ebeam can still be useful if the mirror 306 absorbs larger portions of the ebeam, it is desirable if the ebeam energy loss due to travel through the mirror 306 is as small as possible while still providing desirable reflective properties for incident light (e.g., light having a wavelength in one or more bands of the electromagnetic spectrum from 200 nm to 2000 nm). In many embodiments, it is desirable that the ebeam energy loss as a result of travel through the mirror 306 is less than 5%, desirably less than 2%, more desirably less than 1%, and even less than 0.5%.

Preferred embodiments of mirror 306 are in the form of thin polymer sheets with metallized coatings formed on one or both major faces. Illustrative polymer sheets may have a thickness in the range from 0.001 inches to 0.100 inches. Advantageously, such thin sheets have negligible impact on the ebeam energy while still being strong and durable and while providing excellent reflective properties. In contrast, thin metal sheets in this thickness range tend to be more fragile than might be desired, but still could be used. One suitable mirror embodiment is provided by a polyethylene terephthalate (PET) sheet having a thickness of 0.002 inches and bearing a sputtered aluminum layer on a surface to provide reflectivity.

In the practice of the present invention, one useful way to calculate the impact of a mirror upon ebeam energy is to use the following equation:

$$A = ( \frac{D}{1.4} ) \times ( \frac{T}{1.38} ) \times 99.8$$

wherein A is the percent of the ebeam absorbed by the mirror, D is the density of the sheet in g/ml at 25° C., and T is the sheet thickness in inches. Using the 0.002 inch PET sheet described above, its thickness is 0.002 inches×1.414=0.00283 inches as presented to the ebeam (the sheet is tilted at 45 degrees to the ebeam path), and its density is 1.39 g/ml. Therefore, A is 0.21% to show that such a thin, reflective mirror absorbs a negligible amount of the ebeam energy that pass through mirror 306.

Upper member 300 is secured to lower member 302 in any suitable fashion. According to one technique, using fasteners 316 is suitable. Complementary fastener holes 318 are provided in members 300 and 302 for this purpose. Lower member 302 includes optional window 314 through which optical signals may be projected into the central aperture 312 and redirected by mirror 306 toward the target site 12 (FIG. 1). Using a window 314 is one useful way to provide optical access to the mirror 306. Other strategies are available. For example, the mirror 306 could be mounted to an underside of the assembly 226 where the walls of the assembly 226 would not block optical access to the mirror 306. However, packaging the mirror 306 in the central aperture 312 using window 314 to provide access allows the overall height of the rotary coupling system 95 to be more compact.

FIGS. 5-7, 13-14, 29-30, 32-34, and 45 show the rotary base plate 240 in more detail. Rotary base plate includes upper rim 320 and lower rim 324. Top face 322 is at upper rim 320 and lower face 326 is at lower rim 324. Rotary base plate 240 has shoulder 328 and neck 330. Inner cylindrical wall 332 helps to define central aperture 334 having the common central axis 211 in the assembled coupling system 95. Rotary base plate 240 serves as a main support and mounting member for other components of the second sub-assembly 98.

FIGS. 5-7, 29-30, 32-33, 35, and 42-43 show the rotor 242 in more detail. Rotor 242 includes base 340. Base 340 attaches rotor 242 to the rotary base plate 240. At rotor 242, neck 344 projects upward from base 340. Rotor ring 262 is mounted onto rotor 242 around neck 344. A ring 348 of detent features 349 is formed in top surface 346. Ring 348 is part of rotary indexing and rotary locking systems described further below. Rotor 242 includes recess features to house the outer race 252 of ring bearing 200 as well as the outer race clamp 258. Rotor 242 includes central aperture 350 having the common central axis 211 through which the ebeam 16 (FIG. 1) passes.

FIGS. 5-7, 13-14, 29-30, 32-34, and 45 show the mounting plate 244 in more detail. Mounting plate 244 includes body 360 extending from top rim 362 to bottom rim 366. Top surface 364 is at top rim 362. Top surface 364 is attached to the lower face 326 of the rotary base plate 240. Interior, cylindrical wall 368 defines central aperture 370 having the common central axis 211 through which the ebeam 16 (FIG. 1) travels. The lower face 365 of mounting plate 244 includes rails 372 and slot features (not shown) similar to those on shield 88 in order to couple mounting plate 244 to the head 126 of applicator 86.

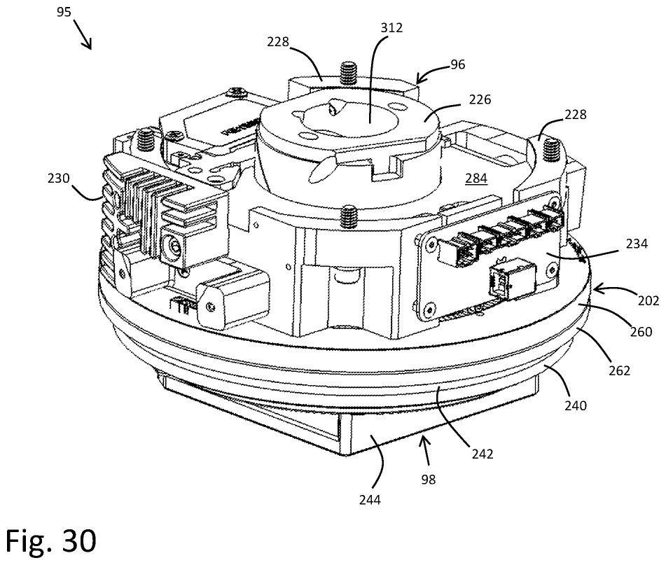

FIGS. 30-31, 33-34, and 41 show the rotary indexing system in more detail formed from plunger assembly 380 mounted on the upper sub-assembly 96 and ring 348 and detent features 349 formed on the rotor 242 of lower sub-assembly 98. Plunger assembly 380 includes a main support plate 382 that is attached to main body 220. Support plate 382 includes a slot 384 in which linear rail 390 is mounted. A carriage 392 rides back and forth along linear rail 390. Mounting holes are used to attach plate 382 to main body 220. Mounting bosses 388 are used to attach guiding frame 394 to the plate 382.

Guiding frame 394 has legs 396 connected at one end by crosspiece 398. Open slot 400 is formed between legs 396 underneath crosspiece 398. Bearing support 402 is attached to sliding carriage 392, and thus can move linearly up and down with the carriage 392. Roller bearing 404 is mounted to the lower end of the bearing support 402. Roller bearing 404 rides in the detent features 349 of detent ring 348. Head 406 of the bearing support 402 fits in the slot 400 to help guide the roller bearing 404 up and down as the bearing 404 rides around ring 348. A spring 403 pushes downward against pocket 408 of bearing support 402 as well as upward against the crosspiece 398 in order to bias roller bearing 404 to be pushed down against the ring 348 while still allowing bearing 404 to move up and down to accommodate the ups and downs of the detent features 349.

In use, the rotary indexing system helps the upper and lower sub-assemblies 96 and 98 to rotate relative to each other in indexed increments corresponding to the number of detent features 349 incorporated into ring 348. Generally, a greater number of detent features 349 provides a greater number of indexed rotational positions as compared to using a lesser number of detent features 349. In one embodiment, using a ring 348 including 180 detent features 349 allowed rotation in two-degree increments.

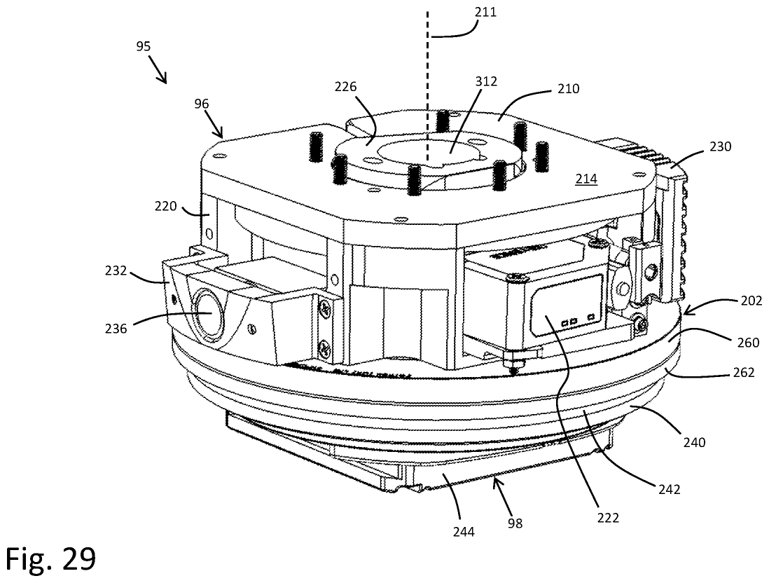

FIGS. 29, 31, 35, 39, and 42-44 show the rotary locking and release mechanism 236 in more detail. Mechanism 236 includes button actuated locking device 432 mounted onto main body 220 of upper sub-assembly 96 and the ring 348 and detent features 349 on the lower sub-assembly 98. The ring 348 and detent features 349 thus play a role both for indexed rotation as well as for rotational locking functionality.

Device 432 includes a housing 434. Slideable locking teeth 436 project from the underside of the housing 434 that faces the ring 348. Housing 434 is deployed so that the slideable locking teeth 436 engage or disengage from ring 348 on demand. The teeth 436 have a sliding range of motion in which the teeth 436 engage with detent features 349 of ring 348. In this configuration, the engaged teeth 436 prevent relative rotation between the sub-assemblies 96 and 98. In effect, rotation of the applicator 86 and shield 88 are locked in this configuration. The slideable locking teeth 436 have a further range of motion in which the teeth 436 can slide radially inward to disengage from the detent features 349 of ring 348. In this configuration, the sub-assemblies 96 and 98 are unlocked and able to rotate relative to each other. In effect, the applicator 86 and shield 88 can rotate in this configuration.

The slideable locking teeth 436 are actuated by pressing or releasing button 438 that is coupled to the locking teeth 436. In an un-pressed, released configuration, the teeth 436 are biased to be engaged with the detent features 349 to lock the rotation. In effect, a locked rotational configuration is the default. A spring or other suitable device can be used to provide the bias to keep the teeth 436 engaged with the detent features 349 when the button 438 is not pressed.

Pushing the button 438 also pushes the teeth 436 radially inward at the same time. This causes the bias against the teeth 436 to be overcome. The teeth 436 slide radially inward to become disengaged from the detent features. This unlocks the rotation, allowing the applicator 86 and shield 88 to be rotated about axis 211. The inward movement of teeth 436 to unlock rotation is shown by arrow 442. Releasing the button 438 allows the bias to push the button 438 outward and the teeth 436 radially outward back into engagement with the detent features 349. The outward move of the teeth 436 back to a locking position is shown by arrow 440. The positioning of teeth 436 is calibrated so that the teeth 436 engage the detent features 349 when the relative rotation of the sub-assemblies 96 and 98 is in an indexed rotational configuration.

FIGS. 5-7, 10, 29-31, 33, 36-37, and 39-40 show the optical illumination system in more detail. The optical illumination system includes at least two illumination functions. First, an illumination source is used to create illumination that is redirected along the ebeam pathway 90 (FIG. 2) in order to illuminate the target site 12 to make it more easily viewed. Second, an illumination source is used to generate a reference mark, such as cross hairs, that is redirected along the ebeam pathway 90 (FIG. 2) along central axis 211 in order to precisely show where the ebeam 16 is aimed. The reference mark is thus projected onto the patient, and a deviation between the projected reference mark and the target site 12 can be compared. This allows the unit 26 to be precisely adjusted to overcome the deviation so that the reference mark is aimed properly at the target site 12.

A support arm 450 serves as a base for the components. Support arm 450 includes mounting bosses 452 for attaching to the main body 220. Laser mounts 456 help to mount laser 454 to the support arm 450. Laser 454 is configured to emit a laser output in the form of a reference mark that can be projected to the target site 12 (FIG. 1). A laser-aiming fixture 458 allows the laser output to be calibrated so that the reference mark is projected to the target site along the center axis 211. An illumination source, such as an LED illumination source 460, generates illumination that also is projected to the target site 12 along the center axis 211. Projecting these along the center axis 211 helps to ensure that projection accuracy is maintained through a suitable range of treatment distances between the end of the shield 88 and the target site 12.

The laser 454 and the illumination source 460 generate optical output from different directions. However, it is helpful to align these so that common components can be used to project the light outputs down to the target site 12. Desirably, the optical signals from the laser 454 and illumination source 460 are redirected accurately down the central axis 211. The combination of the optical signals desirably is accomplished so that the reference marks remain visually observable at the target site 12 rather than being substantially homogenized into a composite illumination in which the reference marks are optically washed out. To this end, optical manifold 476 is provided to receive the illumination and laser reference marks from different directions and then to output the two types of illumination in a common direction.

In one mode of practice, a conventional beam splitter is used in reverse to function as a beam combiner. A beam splitter includes a partially reflective/partially light transmissive element deployed at a 45 degree angle. From one direction, and incident signal can pass straight through the element with only part of that beam being lost to reflection. At the same time, a second signal can enter at 90 degrees from a second direction. Since the surface is partially reflective, a portion of this second signal will be redirected at 90 degrees as an output. The result is that the input signals arrive at the element from two directions but are emitted in the same direction.

For example, consider a beam splitter having a 70R/30T specification. This means that 70% of incident light is transmitted while 30% is reflected. In a desired mode of practice, the LED illumination is aimed so that it enters and leaves the element on a liner path. This means that 70% of the illumination passes through to be projected to the target site 12. In the meantime, the laser signal carrying the reference mark enters the element at a right angle relative to the output direction. This means that 30% of the laser signal is reflected to be projected to the target site 12. The other 70% of the laser signal passes through the element and is blocked with a suitable component such as a neutral density optical filter. This strategy is desired because the laser signal as emitted from the laser is concentrated enough to scatter and create artifacts that could show up at the target site. The strategy described here reduces these scatter and artifact effects.

The optical illumination system also includes an auxiliary mirror 478 on the support arm 450. This auxiliary mirror 478 helps to guide the combined optical signals radially inward with respect to the central core and mirror assembly 226 through the window 314 and toward the mirror 306 so that the light signals can be projected by the mirror 306 downward along the central axis 211 to the treatment site 12. Auxiliary mirror helps to make the overall deployment of the systems 222 and 224 more compact so that the optical signals developed by these systems can be effectively transmitted through window 314 to the mirror 306 and so that the image capturing sensor 474 can appropriately observe the mirror 306 through the window 314.

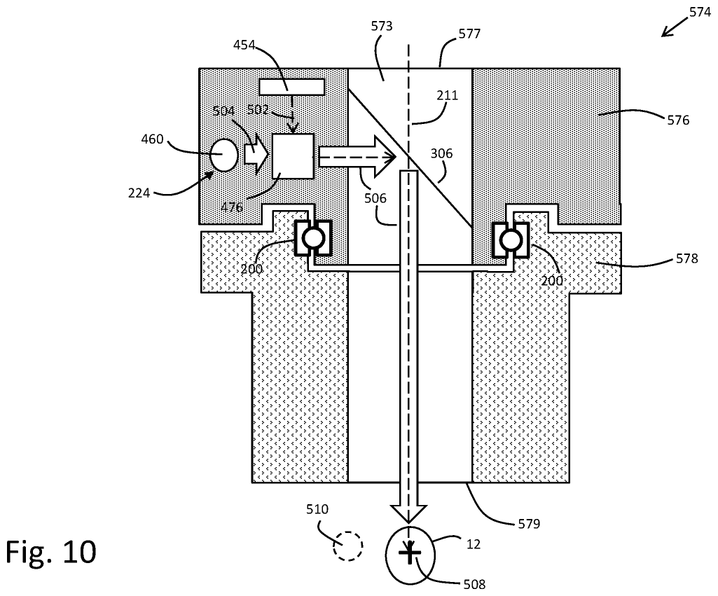

FIGS. 10 and 36-37 schematically how the optical illumination system works. Laser 454 outputs an optical signal 502 that provides a reference mark such as an optical crosshair. One convenient output generates the reference mark from green laser light. An advantage of doing this is that green laser light is easily seen on a variety of different skin tones. Other colors of laser light may be difficult to see for some skin tones. The optical signal 502 of laser 454 is aimed at the optical manifold 476. The optical manifold 476 redirects and emits a portion of the laser optical signal 502 in an output direction that is at 90 degrees relative to the input direction. At the same time, illumination source 460 outputs an illumination signal 504 toward the optical manifold 476. The optical manifold 476 allows a portion of the illumination signal 504 to be emitted in the same output direction as the laser optical signal 502. For purposes of illustration, the two signals transmitted by optical manifold 476 are shown as the optical signal 506. FIG. 10 schematically shows how optical signal 506 is emitted by optical manifold 476 toward the mirror 306. In the more detailed Figures such as FIG. 40, it can be seen that an auxiliary mirror 478 also is used to help direct optical signal 506 to the mirror 306. Mirror 306, being partially reflective to optical illumination, redirects at least a portion of the optical signal 506 along the central axis 211 toward the target site. The result is that an optical reference mark shown as crosshair 508 is projected onto the target site 12 to accurately show where the ebeam 16 is aimed. At the same time, target site 12 is bathed in illumination from the optical signal 506. If the crosshair 508 is not projected onto the target site 12, such as if it shows up as cross hair 510 away from the target site 12, this indicates that ebeam 16 is not properly aimed at target site 12. The visual feedback allows the aim to be easily corrected until the crosshair 508 is in the desired location.

FIGS. 5-7, 9, 10, 29-31, 33, 36-37, and 39-40 show details of the automated detection system. Mounting plate 468 serves as a base for distance sensor 470. Distance sensor 470 is mounted to plate 468. Plate 468 in turn is mounted to main body 220. Distance sensor 470 incorporates a laser source 472 that outputs a laser signal. Distance sensor 470 also incorporates an image capture sensor 474, such as a CMOS sensor.

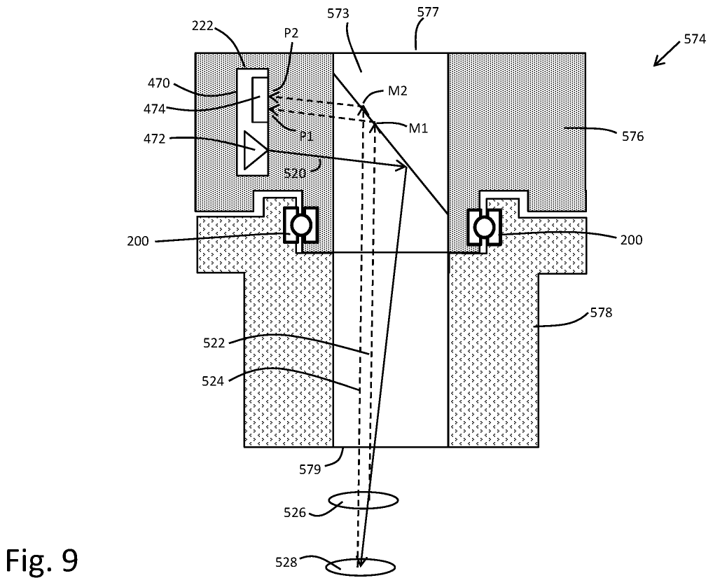

FIGS. 9, and 36-37 schematically show how the automated distance detection system works. The laser emits an output laser signal 520 through window 314 to mirror 306. Mirror 306 reflects the signal 520 downward to the patient surface. At the surface, the laser signal 520 is reflected back up to mirror 306 along a path such as paths 522 or 524. The path of the reflected beam, whether it is path 522, path 524, or another path is a strong function of the distance to the surface generating the reflected beam. For example, path 522 results if the beam 520 is incident upon a relatively close surface 526. In contrast, path 524 results if the beam 520 is incident upon a relative more distant surface 528. In each case, the path 522 or 524 is reflected back onto the mirror 306 at a point M1 or M2 whose location is a function of and is correlated to the distance to the surface 522 or 524, as the case may be. The imaging sensor 474 observes the mirror 306 and captures images of the points M1 or M2, as the case may be, on the image plane as points P1 or P2. The location of P1 or P2 on the image plane differs as a function of distance and is highly correlated to distance. Accordingly, the detection system can use the captured image information to determine the location of the reflected beam in the captured image information and use an appropriate correlation to convert the location into a distance. The distance detection is quite accurate, wherein resultant distance determinations would be accurate to within +/−1 mm or even more accurate such as to +/−0.5 mm or better.

The distance may be computed as between the surface being irradiated and a suitable distance reference on unit 26. One suitable distance reference is to compute the detected distance with respect to the outlet of the scattering foil system 82 (FIG. 2) incorporated into collimator 80. Other locations on unit 26 also may be used as a distance reference if desired. For example, the outlet of window 78 (FIG. 2) may serve as the distance reference. Other alternatives include the outlet of the applicator 86 or shield 88, the outlet of the mounting plate 244, or the like.

FIG. 46 shows an alternative mode of practicing the invention. FIG. 46 is identical to FIG. 7, except that only a single field defining member in the form of shield 88 is attached to the sub-assembly 98 of rotary coupling system 95. Applicator 86 (FIG. 7) is not used. As another difference, the mounting plate 244 is lengthened to help shape the electron beam in the absence of applicator 86.

FIG. 47 shows another mode of practicing the invention. FIG. 47 is identical to FIG. 7 except that only a single field defining member in the form of applicator 88 is attached to the sub-assembly 98 of rotary coupling system 95. Shield 88 (FIG. 7) is not used.

The foregoing detailed description has been given for clarity of understanding only. No unnecessary limitations are to be understood therefrom. The invention is not limited to the exact details shown and described, for variations obvious to one skilled in the art will be included within the invention defined by the claims.

1. An electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface; and
c) a rotary coupling system that rotatably couples at least the first field defining member to an upstream component of the electron beam unit such that the first field defining member is rotatable on demand around a rotational axis independent of rotation of the upstream component, wherein the rotary coupling system comprises a through aperture, an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface.

2. The electron beam radiation system of claim 1, wherein the rotary coupling system comprises a first sub-assembly coupled to the outlet of the electron beam unit and a second sub-assembly rotatably coupled to the first sub-assembly such that the second sub-assembly is rotatable on demand on said rotational axis independent of the first sub-assembly.

3. The electron beam radiation system of claim 1, wherein the electron beam has a beam centerline extending along the linear pathway, and wherein the rotational axis is the same as the beam centerline.

4. The electron beam radiation system of claim 2, wherein the electron beam unit comprises a collimator having an outlet, wherein the collimator outlet is the unit outlet, and wherein the first sub-assembly of the rotary coupling system is coupled to the electron beam unit downstream from the collimator outlet.

5. The electron beam radiation system of claim 2, wherein the rotary coupling system incorporates a rotary encoder that monitors and measures relative rotation between the first and second sub-assemblies of the rotary coupling system.

6. The electron beam radiation system of claim 1, wherein rotational axis of the rotary coupling system is co-linear and coincident with the central axis of the electron beam linear pathway.

7. The electron beam radiation system of claim 1, further comprising a mirror in the through aperture of the rotary coupling system, wherein the mirror is at least partially transparent to the electron beam and is positioned such that the electron beam passes through the mirror as the electron beam travels along the linear pathway through the through aperture of the rotary coupling system.

8. The electron beam radiation system of claim 7, wherein the mirror is tilted at a non-parallel and non-orthogonal angle relative to the central axis of the electron beam linear pathway.

9. The electron beam radiation system of claim 7, wherein the mirror is at least partially reflective to visible light at one or more wavelength bands in a range from 430 nm to 750 nm.

10. The electron beam radiation system of claim 7, wherein the mirror comprises a polymer sheet having first and second major faces and having a metallized coating on one or both major faces.

11. The electron beam radiation system of claim 7, wherein the mirror comprises a polyethylene terephthalate sheet.

12. The electron beam radiation system of claim 11, further comprising an aluminum layer provided on the polyethylene terephthalate sheet in a manner to provide reflectivity.

13. The electron beam radiation system of claim 7, further comprising a body positioned in the through aperture of the rotary coupling system, said body including an upstream member and a downstream member, and wherein the mirror is clamped in place between the upstream and downstream members at an interface between the upstream and downstream members.

14. The electron beam radiation system of claim 7, wherein the rotary coupling system comprises a window through which light can be directed at the mirror in a manner such that the mirror re-directs the light to a target site on the surface.

15. The electron beam radiation system of claim 14, further comprising an illumination source outside the rotary coupling system that provides illumination through the window that is redirected by the mirror to illuminate the target site.

16. The electron beam radiation system of claim 14, further comprising an illumination source outside the rotary coupling system that provides an optical signal through the window that is redirected by the mirror in a manner that projects a reference mark onto the surface.

17. The electron beam radiation system of claim 14, wherein the mirror re-directs the illumination onto the surface along the central axis of the electron beam linear pathway.

18. The electron beam radiation system of claim 16, wherein the mirror re-directs the optical signal along the central axis of the electron beam linear pathway such that the reference mark is projected onto the surface in a manner to show where the electron beam is aimed at the surface.

19. The electron beam radiation system of claim 14, further comprising an illumination source, a laser, and an optical manifold that are positioned outside the window, wherein the illumination source provides illumination that is received by the optical manifold, wherein the laser provides an optical laser signal including a laser reference mark that is received by the optical manifold, and wherein the optical manifold combines the illumination and the optical laser signal and directs the combined illumination and optical laser signal through the window to the mirror such that the mirror re-directs and projects the combined illumination and optical laser signal to the surface.

20. The electron beam radiation system of claim 16, wherein the optical signal comprises green laser light.

21. The electron beam radiation system of claim 19, wherein the optical manifold redirects and emits the optical laser signal in an output direction that is 90 degrees relative to the input direction of the optical laser signal received by the optical manifold.

22. The electron beam radiation system of claim 14, further comprising a distance sensor, wherein the distance sensor comprises:
a laser positioned outside the window that outputs a laser signal through the window such that the mirror redirects the laser signal to the surface and such that the laser signal is reflected from the surface back onto a reflection point on the mirror, and
an imaging device that observes the mirror and captures an image of the reflection point, wherein the location of the reflection point on an image plane of the imaging device is correlated to the distance of the surface from a distance reference.

23. An electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface; and
ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
iii) a window through which light can be directed at the tilted mirror from a location outside the through aperture of the rotary coupling system; and
d) a light system positioned outside the through aperture of the rotary coupling system, wherein the light system produces a light signal and emits the light signal in a manner such that the light signal comprises light from one or more wavelength bands of the electromagnetic spectrum in the range from 200 nm to 2000 nm and is aimed at the tilted mirror through the window in a manner effective to be reflected downstream by the mirror along the linear pathway toward the surface.

24. An electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
iii) a window through which at least one optical signal can be directed at the tilted mirror from a location outside the through aperture of the rotary coupling system; and
d) a light system positioned outside the through aperture of the rotary coupling system, wherein the light system produces a light signal and emits the light signal in a manner such that the light signal is aimed through the window at the tilted mirror in a manner effective to be reflected downstream along the linear pathway to the surface through the first field defining member through aperture.

25. The system of claim 24, wherein the light system comprises an LED light source that produces at least a portion of the light signal in a manner such that the LED light reflected downstream through the first field defining member outlet illuminates the surface with illumination comprising LED light from one or more wavelength bands of the electromagnetic spectrum in the range from 200 nm to 2000 nm.

26. An electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
iii) a window through which light can be directed at the tilted mirror from a location outside the through aperture of the rotary coupling system; and
d) a light system positioned outside the through aperture of the rotary coupling system, wherein the light system produces a light signal and emits the light signal in a manner such that the light signal is aimed at the tilted mirror through the window in a manner effective to be reflected downstream along the linear pathway through the first field defining member to the surface, wherein the light system comprises a laser light source that produces a light signal comprising a visually observable optical reference mark that is reflected downstream through the first field defining member outlet onto the surface in a manner such that the location of the reference mark on the surface is indicative of how the electron beam is aimed at the surface.

27. An electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising a central axis, an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
iii) a window through which at least one optical signal can be directed at the tilted mirror from a location outside the through aperture of the rotary coupling system; and
d) a light system positioned outside the through aperture of the rotary coupling system, wherein the light system produces a composite light signal and emits the composite light signal in a manner such that the composite light signal is aimed at the tilted mirror in a manner effective to be reflected downstream along the linear pathway through the first field defining member toward the surface, wherein the light system comprises:
i) a laser light source that produces at least a portion of a first light signal comprising a visually observable optical reference mark.
ii) an LED light source that produces at least a portion of a second light signal comprising visually observable LED illumination; and
ii) an optical combiner that combines at least the first and second light signals to provide the composite light signal in a manner such that the reference mark is reflected downstream through the first field defining member onto the surface in a manner such that the location of the reference mark on the surface is indicative of how the electron beam is aimed at the surface and such that the LED illumination illuminates the surface where the electron beam is aimed.

28. An electron beam radiation system that emits an electron beam at a surface, comprising:
a) an electron beam unit having a unit outlet, wherein the electron beam unit produces the electron beam and emits the electron beam from the unit outlet on a linear pathway leading from the unit outlet to the surface, wherein the linear pathway has a central axis;
b) at least a first field defining member positioned on the linear pathway downstream from the unit outlet, wherein the first field defining member has a through aperture comprising an inlet through which the electron beam enters the first field defining member through aperture as the electron beam travels along the linear pathway to the surface, and an outlet through which the electron beam leaves the first field defining member through aperture as the electron beam travels along the linear pathway to the surface;
c) a rotary coupling system that rotatably couples at least the first field defining member to an upstream component of the electron beam unit such that the first field defining member is rotatable on demand around a rotation axis independent of rotation of the upstream component, wherein the rotary coupling system comprises:
i) a through aperture comprising an inlet through which the electron beam enters the rotary coupling system through aperture as the electron beam travels along the linear pathway to the surface, and an outlet through which the electron beam leaves the rotary coupling system through aperture as the electron beam travels along the linear pathway to the surface;
ii) a tilted mirror mounted at a tilted angle in the through aperture of the rotary coupling system, wherein the mirror is tilted at a non-parallel and non-orthogonal angle relative to the linear pathway, wherein the mirror is at least partially reflective with respect to optical illumination in one or more wavelength bands of the electromagnetic spectrum in a range from 200 nm to 2000 nm, and wherein the tilted mirror is at least partially transparent to the electron beam such that at least a portion of the electron beam passes through the tilted mirror as the electron beam travels along the linear pathway; and
iii) a window through which light can be directed at the tilted mirror from a location outside the through aperture of the rotary coupling system; and
d) a distance detection system positioned outside the through aperture of the rotary coupling system, wherein the distance detection system comprises a controller, a laser light source, and an image capturing sensor, wherein:
the laser light source is configured to emit a laser light signal at the tilted mirror in a manner effective to be reflected downstream along the linear pathway through the first field defining member toward the surface such that at least a portion of the laser light signal is reflected from the surface back to a location on the tilted mirror that is a function of a distance characteristic of the surface relative to a distance reference; and
the image capturing sensor observes and captures image information of the tilted mirror, said image information indicative of the location on the tilted mirror onto which the laser light signal is reflected from the surface; and
the control system uses the capture image information to determine a distance characteristic of the surface with respect to the distance reference.
